(19) United States
(12) Patent Application Publication
(10) Pub. No.: US 2023/0293614 A1
(43) Pub. Date: Sep. 21, 2023

(54) COMPOSITIONS AND METHODS OF TREATING UPS-ASSOCIATED DISEASES

(71) Applicant: Board of Supervisors of Louisiana State University and Agricultural and Mechanical College, Baton Rouge, LA (US)

(72) Inventors: Elizabeth FLOYD, Baton Rouge, LA (US); Adam MELVIN, Baton Rouge, LA (US)

(21) Appl. No.: 18/016,189

(22) PCT Filed: Jul. 13, 2021
(86) PCT No.: PCT/US21/41444

§ 371 Date: Jan. 13, 2023

Related U.S. Application Data
(60) Provisional application No. 63/051,327, filed on Jul. 13, 2020.
(60) Provisional application No. 63/094,642, filed on Oct. 21, 2020.

(51) Int. Cl. A61K 36/282; A61K 45/06; A61K 31/045; A61P 35/00

(52) U.S. Cl. CPC A61K 36/282; A61K 45/06; A61K 31/045; A61P 35/00

(57) ABSTRACT

This invention is directed to methods of treating disease characterized by aberrations in the ubiquitin proteasome system (UPS).

This application is a National Stage Entry of PCT/US21/41444, filed Jul. 13, 2021 which claims priority from U.S. Provisional Patent Application No. 63/051,327, filed on Jul. 13, 2020, and U.S. Provisional Patent Application No. 63/094,642, filed on Oct. 21, 2020, the contents of which are incorporated by reference herein in their entireties.

### GOVERNMENT INTERESTS

This invention was made with government support under Grant No. P50AT002776, Grant No. R03EB02935, and Grant No. U41AT008706 awarded by the National Institutes of Health; and Grant No. CBET1509713 awarded by the National Science Foundation. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Aug. 30, 2021, is named 2932719-124WO1_SL.txt and is 3,752 bytes in size.

### FIELD OF THE INVENTION

This invention is directed to compositions and methods for treating diseases characterized by aberrations in the ubiquitin proteasome system (UPS).

### BACKGROUND OF THE INVENTION

Aberrations in the ubiquitin proteasome system (UPS) contribute to the pathological states of several clinical disorders, such as cancer. A significant challenge with treating cancer is the heterogeneity associated with cancer cells.

### SUMMARY OF THE INVENTION

Aspects of the invention are drawn to a method of treating a disease or disorder characterized by aberrations in the ubiquitin proteasome system (UPS). For example, the disease or disorder can comprise a cell proliferative disease or disorder, a muscle wasting disease or disorder, a renal disease or disorder, an inflammatory disease or disorder, a neurodegenerative disease or disorder, or a ubiquitin-proteasome system (UPS)-associated disease or disorder. For example, the cell proliferative disease or disorder can be cancer, such as multiple myeloma, kidney cancer, breast cancer, lung cancer, brain cancer, skin cancer, liver cancer, liposarcoma, and pancreatic cancer. For example, the muscle wasting disease or disorder can be sarcopenia, muscular atrophy, cardiac atrophy, cachexia, amyotrophic lateral sclerosis (ALS), muscular dystrophy, multiple sclerosis (MS), and spinal muscular atrophy. For example, the UPS-associated disease or disorder can be characterized by aberrations in the ubiquitin-proteasome system (UPS).

In embodiments, the method can comprise administering to a subject a therapeutically effective amount of a botanical extract or components thereof, wherein the botanical extract is isolated from Artemisia spp. In embodiments, the botanical extract can be isolated from Artemisia dracunculus L. In embodiments, the botanical extract can comprise PMI5011 or a component thereof. For example, the component can be DMC-1, DMC-2, davidigenin, sakuranetin, or 6-demethoxycapillarisin. In embodiments, the botanical extract can comprise a knockout extract (KOE). In embodiments, the KOE can have reduced levels of at least one component relative to the normal extract. For example, the KOE can have reduced levels of at least one of DMC-1 or DMC-2, relative to the normal extract. For example, the KOE does not comprise DMC-1 or DMC-2. For example, the KOE does not comprise DMC-1 and DMC-2. In embodiments, the botanical extract can comprise an alcoholic extract. For example, the alcoholic extract can comprise an ethanolic extract. In embodiments, the botanical extract can modulate the activity of at least one deubiquitinating enzyme (DUB).

In embodiments, the botanical extract can be administered to a subject orally, intravenously, sub-cutaneously, or transdermally.

Embodiments can further comprise administering to a subject in need thereof one or more additional active agents. For example, the one or more additional active agents can comprise one or more anti-cancer agents, one or more anti-inflammatory agents, or one or more neuroprotective agents. For example, the one or more anti-cancer agents can comprise Marizomib, Ixazomib, Borezomib, or Carfilzomib.

Aspects of the invention are also drawn towards a therapeutic preparation comprising a botanical extract isolated from Artemisia spp., wherein the botanical extract modulates the activity of the UPS. In embodiments, the botanical extract modulates the activity of at least one deubiquitinating enzyme (DUB). In embodiments, the botanical extract can be isolated from Artemisia dracunculus L. In embodiments, the botanical extract can comprise PMI5011 or a component thereof. For example, the component can be DMC-1, DMC-2, davidigenin, sakuranetin, or 6-demethoxycapillarisin. In embodiments, the botanical extract can comprise a knockout extract (KOE). In embodiments, the KOE can have reduced levels of at least one component relative to the normal extract. For example, the KOE can have reduced levels of at least one of DMC-1 or DMC-2, relative to the normal extract. For example, the KOE does not comprise DMC-1 or DMC-2. For example, the KOE does not comprise DMC-1 and DMC-2. In embodiments, the botanical extract can comprise an alcoholic extract. For example, the alcoholic extract can comprise an ethanolic extract.

Further, aspects of the invention are drawn towards a drug-screening method. In embodiments, the method can comprise culturing a population of cells; incubating the population of cells with a botanical extract or component thereof, wherein the botanical extract is isolated from Artemisia spp; and determining the effect of the botanical extract or component thereof on UPS activity. Embodiments can further comprise a step of obtaining a population of cells from a subject. Embodiments can further comprise a step of culturing a population of control cells. Embodiments can further comprise a step of administering the botanical extract or component thereof to a subject. In embodiments, the population of cells can comprise a population of cancer cells. For example, the cancer cells can comprise MM.1S cells or OPM2 cells. In embodiments, the population of cells are incubated with an amount of the botanical extract for a period of time. In embodiments, the UPS activity comprises DUB activity.

An aspect of the present invention provides a method of treating disease characterized by aberrations in the ubiquitin proteasome system. In an embodiment, the invention is drawn towards a method of treating a disease or disorder characterized by aberrations in the ubiquitin proteasome system (UPS), the method comprising administering to a subject a therapeutically effective amount of an extract isolated from Artemisia dracunculus L. In another embodiment, the invention is directed towards a method of treating cell proliferative disease or disorder, the method comprising administering to a subject a therapeutically effective amount of an extract isolated from Artemisia dracunculus L.

In an embodiment, the isolated extract comprises a knockout extract (KOE). For example, the KOE is deficient in or completely lacks at least one of DMC-1, DMC-2.

In an embodiment, the isolated extract comprises PMI5011 or an isolated component thereof. In a further embodiment, the isolated component thereof comprises DMC-1, DMC-2, davidigenin, sakuranetin, 6-demethoxycapillarisin, or any combination thereof.

In an embodiment, the extract comprises an alcoholic extract. In a further embodiment, the extract comprises an ethanolic extract.

In another embodiment, the isolated extract modulates the activity of at least one deubiquitinating enzyme (DUB). In another embodiment, the disease or disorder comprises a UPS associated disease or disorder that is linked to deubiquitinase activity.

In another embodiment, the disease comprises a cancer. In a further embodiment, the cancer comprises multiple myeloma.

In another embodiment, the disease or disorder comprises sarcopenia or renal diseases or disorders. In another embodiment, the extract is administered to a subject orally, intravenously, sub-cutaneously, or transdermally.

Other objects and advantages of this invention will become readily apparent from the ensuing description.

### BRIEF DESCRIPTION OF THE FIGURES

FIG. 1 shows effect of PMI5011 and its components on MM.1S cellular viability. Model multiple myeloma MM.1S cells were exposed to respective treatments (PMI5011, DMC-1, DMC-2, DVG, KOE and control DMSO) overnight for 16 h before testing for cellular viability using a standard colorimetric MTT assay. Normalized S: N values were obtained from treated cellular absorbance and background control at λₑₓ=544 nm and λₑₘ=90 nm. PMI5011 and its individual components did not have a significant effect on cellular viability (p>0.05).

FIG. 2 shows effect of PMI5011 on DUB activity in model cell lines. A) C2C12 cells B) MM.'S cells and C) OPM-2 cells were treated with DMSO (control) and 10 μg/mL PMI5011 (treatment) for 16 h before measuring the DUB [E] activity with Z-LRGG-AMC [S] (SEQ ID NO: 1). The rate values were obtained by measuring the fluorescence readout of free AMC [P] as a result of DUB activity at different time points for different [S] concentrations. *p<0.01 and **p<0.0001 favoring the alternate hypothesis V_{control}≠V_{PMI5011}.

FIG. 3 shows effect of bioactive components of PMI5011 on DUB activity. MM.1S cells (i) or OPM-2 cells (ii) were treated with DMSO (control) and 1,10,30 μg/mL of A) DMC-1, B) DMC-2, C) DVG, D) KOE (treatment) for 16 h before measuring the DUB [E] activity with Z-LRGG-AMC [S] (SEQ ID NO: 1). The rate values were obtained by measuring the fluorescence readout of free AMC [P] as a result of DUB activity at different time points for different [S] concentrations. *p<0.01 and **p<0.0001 favoring the alternate hypothesis V_{control}≠V_{PMI5011}.

FIG. 4 shows bar graph representing the effect of PMI5011 and its bioactive components on DUB activity in i) MM.1S and ii) OPM2 cell extracts. The maximum rate of the reaction for respective treatment compound (Vₘₐₓ) was normalized with the maximum rate of the reaction for control DMSO (V_{max,c}). The blue dashed line represents the line of normalization with control.

FIG. 5 shows effect of increasing concentrations of different compounds i) DMC1, ii) DMC2 iii) DVG iv) KOE on DUB activity in A) MM.1S and B) OPM-2. The normalized rates Vₘₐₓ for different concentrations of each of the compound was plotted and linear/non-linear regression curve was fitted for each of the plot.

FIG. 6 shows graph of rates components 1 and 2.

FIG. 7 shows graph of control and 5011 treated lysates.

FIG. 8 shows graph of control, 5011 treated lysates, 25 uM IU1, 50 uM IU1, and KOE treated lysates.

FIG. 9 shows graph of better fitting curve where values at 100 μM and 125 μM.

FIG. 10 shows graph of control and 5011 treated lysates.

FIG. 11 shows graph of control, lysates, 1 μg/mL DMC, 10 μg/mL DMC, and 30 ng/mL DMC treated lysates.

FIG. 12 shows the effect of PMI5011 and its components on OPM2 cellular viability. Model multiple myeloma OPM2 cells were exposed to respective treatments (PMI5011, DMC-1, DMC-2, DVG, KOE, and control DMSO) overnight for 16 h before testing for cellular viability using a standard colorimetric MTT assay. Normalized S:N values were obtained from treated cellular absorbance and background control at λₑₓ=544 nm and λₑₘ=90 nm. PMI5011 and its individual components did not have a significant effect on cellular viability (p>0.05).

FIG. 13 shows graphs of the effect of PMI-5011 treatment on DUB activity in model mammalian cell lines. Panel A) C2C12 cells Panel B) MM.1S cells and Panel C) OPM2 cells were treated with DMSO (control) or 10 μg/mL PMI-5011 (treatment) for 16 h before measuring the DUB activity. The rate values were obtained by measuring the fluorescence readout of free AMC as a result of DUB activity at different time points for different [S] concentrations. *p<0.01 and **p<0.0001 favoring the alternate hypothesis V_{control}≠V_{PMI-5011}. Figure discloses “LRGG” as SEQ ID NO: 1.

FIG. 14 shows graphs of minimal cytotoxic effect of PMI-5011 and selected bioactive compounds on cellular viability. MM.1S (Panel A) and OPM2 (Panel B) cells were exposed to respective treatments (PMI-5011, DMC-1, DMC-2, Davidigenin, KOE) and a negative control EtOH or 16 h before testing for cellular viability using an MTT assay. Percentage cytotoxicity values were obtained by measuring absorbance values of positive control vehicle (DMSO), treatment and background control samples at λₑₓ=544 nm and λₑₘ=590 nm. * denotes p<0.01 for all the treatment samples when compared to the negative control EtOH, demonstrating that PMI-5011 and selected bioactive compounds did not have a negative effect on MM.1S and OPM2 cellular viability. All data are representative of duplicate experiments with each data point performed in triplicate to produce the error bars.

FIG. 15 shows graphs of effect of selected bioactive compounds on deubiquitinating enzyme activity. MM.1S cells and OPM2 cells were treated with DMSO (control, grey) or 1 μg/mL (black), 10 μg/mL (black—dashed), or 30 μg/mL (black—dotted) of DMC-1 (Panel A, Panel E), DMC-2 (Panel B, Panel F), DVG (Panel C, Panel G), or KOE (Panel D, Panel H) for 16 h before measuring the DUB [E] activity with Z-LRGG-AMC [S] (SEQ ID NO: 1). The rate values were obtained by measuring the fluorescence readout of free AMC [P] as a result of DUB activity at different time points for different [S] concentrations. *p<0.01 and **p<0.0001 favoring the alternate hypothesis V_{control}≠V_{PMI-5011}. Davidigenin is denoted as DVG.

FIG. 16 shows graphs of quantification of the effect of PMI-5011 and selected bioactive components on DUB activity in MM.1S (Panel A) and OPM2 (Panel B) cell extracts. The maximum rate of the reaction for respective treatment compound (Vₘₐₓ) was normalized with the maximum rate of the reaction for control DMSO (V_{max,c}). The blue dashed line represents the line of normalization with control. Normalized reaction rate values above the blue dashed line represents enhancement of DUB activity while values below this line represents inhibition of DUB activity.

FIG. 17 shows graphs of the effect of increasing concentrations of the selected bioactive compounds. Normalized reaction rates for cells treated with DMC-1 (Panel A, Panel E), DMC-2 (Panel B, Panel F), DVG (Panel C, Panel G) or KOE (Panel D, Panel H) were compared to investigate DUB activity in MM.1S and OPM2 cells. The normalized reaction rates Vₘₐₓ for different concentrations of each of the compound was plotted and linear/non-linear regression curve (blue dashed line) was fitted for each of the plot. Linear increase and decrease (DMC-1, DMC-2, DVG) and an exponential increase (KOE) were observed in MM.1S cells while, a contrasting trend with an exponential increase (DMC-1, DMC-2, DVG) or a linear decrease (KOE) was observed in OPM2 cells.

FIG. 18 shows a graph of mRNA profiling for deubiquitinating enzyme genes in MM.1S and OPM2 multiple myeloma cell lines. mRNA profiling for standard deubiquitinase genes in MM.1S and OPM2 reveals a differential expression of deubiquitinating enzyme levels in these two cell lines. The fold changes were obtained by normalizing all the gene levels with the least expressed USP15 in MM.1S. *p<0.01 and **p<0.0001 favoring the alternate hypothesis of fold change in gene levels not significantly equal to control MM.1S's USP15 against the null hypothesis of fold change in gene levels significantly equal to control MM. 1S's USP15.

FIG. 19 shows Panel A) HPLC chromatogram and Panel B) mass spectrum of pure DMC-1.

FIG. 20 shows Panel A) ¹³C NMR spectrum and Panel B) 1H NMR spectrum of pure DMC-1.

FIG. 21 shows Panel A) HPLC chromatogram and Panel B) mass spectrum of pure DMC-2.

FIG. 22 shows Panel A) ¹³C NMR spectrum and Panel B) ¹H NMR spectrum of pure DMC-2.

FIG. 23 shows Panel A) HPLC chromatogram and Panel B) mass spectrum of pure davidigenin.

FIG. 24 shows Panel A) ¹³C NMR spectrum and Panel B) ¹H NMR spectrum of pure davidigenin.

FIG. 25 shows MM.1S and OPM2 cells exhibit different USP7 activity in the presence of a commercial inhibitor. Concentration-dependent DUB-mediated cleavage rates of the Z-LRGG-AMC (SEQ ID NO: 1) reporter were evaluated in MM.1S (Panel A) and OPM2 (Panel B) cell lines. Cells were pretreated with 1 μM HBX or DMSO for 24 h before performing the enzyme assay. Legends are the same in all plots. Rate data demonstrate Michaelis-Menten kinetics (r²>0.99) in all cases. (Panel C) Michaelis-Menten kinetics parameters calculated for the DMSO and HBX curves. * denotes p<0.001. (Panel D) ANOVA F-statistics on cell dependent expression of USP7. p>0.05 favors the null hypothesis: V_{MM.1S}=V_{OPM2} or Vₘₐₓ/K_{m, MM.1S}=Vₘₐₓ/K_{m, OPM2} while p<0.05 favors the alternate hypothesis V_{MM.1S}≠V_{OPM2} or Vₘₐₓ/K_{m, MM.1S}≠Vₘₐₓ/K_{m, OPM2}.

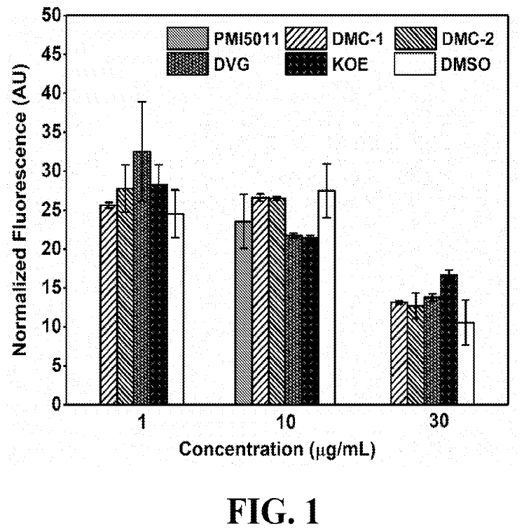

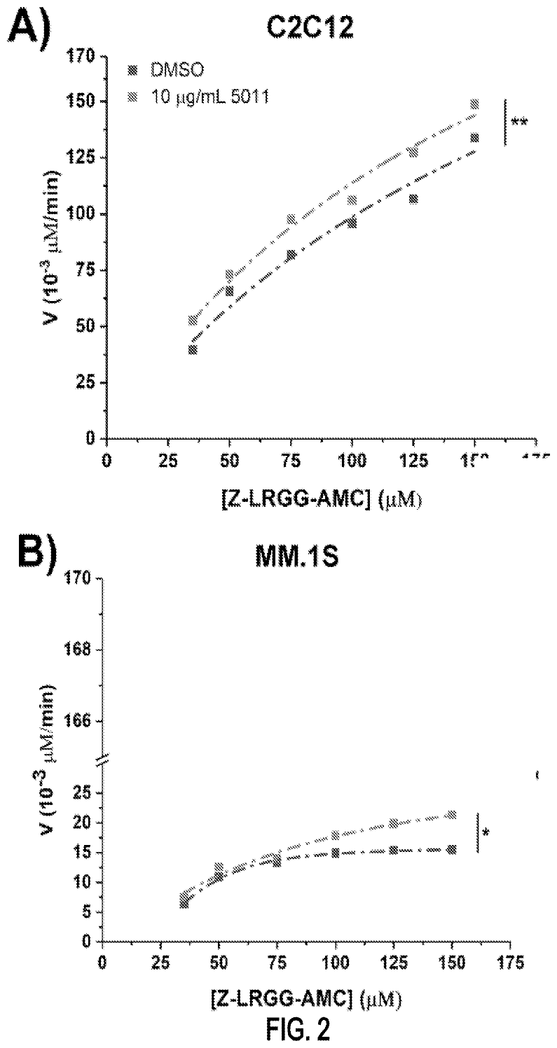

### DETAILED DESCRIPTION OF THE INVENTION

Aberrations in the ubiquitin proteasome system (UPS) contribute to the pathological states of several clinical disorders, such as cancer. While inhibition of the proteasome has proven to be effective in the treatment of multiple myeloma (MM), a significant challenge with these proteasome-targeted therapeutics is the heterogeneity associated with cancer cells.

The invention described herein is directed to methods of treating diseases characterized by aberrations in the ubiquitin proteasome system (UPS), such as cell proliferation diseases, by administering a plant extract. For example, a plant extract, such as an extract isolated from Artemisia dracunculus L., is administered to treat a disease characterized by aberrations in the ubiquitin proteasome system (UPS), such as a cell proliferation disease.

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Methods of Treating a Disease

The ubiquitin-proteasome system (UPS) is a well-controlled biochemical pathway for the recognition and degradation of misfolded, damaged or dysregulated proteins.¹³ The pathway requires a concerted action of a series of enzymes: E1 ubiquitin activating enzymes, E2 ubiquitin conjugating enzymes and E3 ubiquitin ligases, responsible for attaching a polyubiquitin chain to a lysine residue on a target protein, thus altogether marking them for proteasomal degradation. While the above enzymes aid in the conjugation of ubiquitin to substrates, there are also dozens of other enzymes called the deubiquitinating enzymes (DUBs), that can reverse the process by detaching the polyubiquitin chain to rescue the proteins from degradation.

Thus, the degradation of activity of the UPS calibrates the abundance of intracellular proteins not only for homeostatic regulation, but also to mediate critical changes in metabolism in response to an external signal or for cell cycle progression. Aberrations in the UPS contributes to the pathological states of several clinical disorders including inflammation, neurodegeneration and cancer. Human cancer cells possess elevated levels of proteasome activity and are more sensitive to proteasome inhibitors than normal cells. Targeting proteolytic and regulatory components of the UPS can be an efficient strategy for cancer treatment. For example, inhibition of the proteasome has proven to be effective in the treatment of multiple myeloma (MM).

Aspects of the invention are drawn towards methods of treating a disease characterized by aberrations in the ubiquitin proteasome system (UPS). Aberrations in the UPS can refer to modifications, changes and alterations in the ubiquitin proteasome system. For example, aberrations in the UPS can comprise alternations in the protein sequence of oncoproteins or enzymes of the ubiquitin system due to gene mutations that result in abnormal increases in protein abundance.

For example, embodiments are drawn towards methods of treating muscle loss caused by catabolic disease or disorder. A “catabolic disease or disorder” can refer to a condition characterized by rapid weight loss and loss of fat and skeletal muscle mass, which can occur in a background of either an acute, self-limited disease (such as injury, infection) or a chronic condition (such as multisystem organ failure, advanced cancer, chemotherapy. The catabolic disease or disorder can be characterized by aberrations in metabolic activity concerned with the breakdown of molecules for the release of energy. Non-limiting examples of the catabolic disease or disorder comprises sarcopenia, renal diseases, or cancer. In an embodiment, the disease comprises a UPS associated disease that is linked to deubiquitinase activity.

Embodiments are drawn towards methods of treating muscle wasting diseases or disorders. The term “muscle wasting disease” can refer to diseases that occur due to muscle weakness. Non-limiting examples of muscle wasting diseases or disorders that can be treated by embodiments described herein comprise sarcopenia, muscular atrophy, cardiac atrophy, cachexia, amyotrophic lateral sclerosis (ALS), muscular dystrophy, multiple sclerosis (MS), and spinal muscular atrophy.

Embodiments are also drawn towards methods of treating a cell proliferative disease or disorder. A “cell proliferative disease or disorder” or can refer to a disease or disorder wherein unwanted cell proliferation of one or more subset(s) of cells in a multicellular organism occurs, resulting in harm to the multicellular organism. For example, the cell proliferative disease or disorder is cancer.

The terms “cancer” and “cancerous” can refer to or describe the physiological condition in mammals that is typically characterized by unregulated cell growth. Examples of cancer include, but are not limited to, blood-borne cancers (e.g., multiple myeloma, lymphoma and leukemia), and solid cancers.

In embodiments, the cancer can comprise those that are metastatic or are not metastatic or are metastatic.

In embodiments, the cancer can include, but is not limited to, solid cancer and blood borne cancer.

Examples of cancers can include, but not be limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastasis, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4 colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

The term “tumor” can refer to all neoplastic cell growth and proliferation, whether malignant or benign, and all pre-cancerous and cancerous cells and tissues. “Neoplastic,” as used herein, can refer to any form of dysregulated or unregulated cell growth, whether malignant or benign, resulting in abnormal tissue growth. Thus, “neoplastic cells” can include malignant and benign cells having dysregulated or unregulated cell growth.

“Blood borne cancer” or “hematologic malignancy” can refer to cancer of the body's blood-forming and immune system—the bone marrow and lymphatic tissue. Such cancers include leukemias, lymphomas (Non-Hodgkin's Lymphoma), Hodgkin's disease (also called Hodgkin's Lymphoma) and myeloma. In one embodiment, the myeloma is multiple myeloma. In some embodiments, the leukemia is, for example, acute myelogenous leukemia (AML), acute lymphocytic leukemia (ALL), adult T-cell leukemia, chronic lymphocytic leukemia (CLL), hairy cell leukemia, myelodysplasia, myeloproliferative disorders, chronic myelogenous leukemia (CML), myelodysplastic syndrome (MDS), human lymphotropic virus-type 1 (HTLV-1) leukemia, mastocytosis, or B-cell acute lymphoblastic leukemia. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin lymphoma (HL), non-Hodgkin lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma.

The major types of cancer are carcinomas, sarcomas, melanomas, lymphoma, and leukemias. Carcinomas originate in the skin, lungs, breasts, pancreas, and other organs and glands. Lymphomas are cancers of lymphocytes. Leukemia is cancer of the blood. It does not usually form solid tumors. Sarcomas arise in bone, muscle, fat, blood vessels, cartilage, or other soft or connective tissues of the body. Melanomas are cancers that arise in the cells that make the pigment in skin. Non-limiting examples of cancers include ovarian cancer, breast cancer, lung cancer, prostate cancer, cervical cancer, pancreatic cancer, colon cancer, stomach cancer, esophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, bone marrow cancer, lymphoma cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma, lymphoma, and leukemia. In an embodiment, the cancer comprises one or more of a colon cancer, colorectal cancer, gastro-intestinal cancer, breast cancer, bladder cancer, kidney cancer, leukemia, brain cancer, sarcoma, astrocytoma, acute myelogenous leukemia (AML), and diffuse large B-lymphoma.

In embodiments, the cancer is multiple myeloma. For example, the DUBs can overcome resistance to proteasome inhibitors in treating multiple myeloma. Non-limiting examples of cancers that can be treated by embodiments described herein comprise multiple myeloma, kidney cancer, breast cancer, lung cancer, brain cancer, skin cancer, liver cancer, liposarcoma, and pancreatic cancer.

Embodiments are also drawn towards methods of treating inflammatory diseases or disorders. An “inflammatory disease or disorder” can refer to a disease or disorder characterized by chronic or acute inflammation. Numerous inflammatory diseases are known in the art, such as arthritis, including rheumatoid arthritis, osteoarthritis, psoriatic arthritis, juvenile idiopathic arthritis; necrotizing enterocolitis (NEC); gastroenteritis; intestinal flu; stomach flu; pelvic inflammatory disease (PID); emphysema; pleurisy; pyelitis; pharyngitis; sore throat; angina; acne vulgaris; rubor; urinary tract infection; appendicitis; bursitis; colitis; cystitis; dermatitis; phlebitis; rhinitis; tendonitis; tonsillitis; vasculitis; asthma; autoimmune diseases; celiac disease; chronic prostatitis; glomerulonephritis; hypersensitivities; inflammatory bowel diseases; pelvic inflammatory disease; reperfusion injury; sarcoidosis; transplant rejection; vasculitis; interstitial cystitis; hay fever; periodontitis; atherosclerosis; psoriasis; ankylosing spondylitis; juvenile idiopathic arthritis; Behcet's disease; spondyloarthritis; uveitis; systemic lupus erythematosus, and some cancers (e.g., gallbladder carcinoma). In embodiments, the inflammatory disease or disorder comprises asthma, arthritis, diabetes, heart disease, fatty liver disease, endometriosis, and inflammatory bowel disease.

Embodiments are also drawn towards methods of treating neurodegenerative diseases or disorders. The term “neurodegenerative disease or condition” can refer to a disease or condition in which the function of a subject's nervous system becomes impaired. Non-limiting examples of neurodegenerative diseases that can be treated with an extract or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, epilepsy, Friedreich ataxia, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK5), Parkinson disease 9 (PARK5), Parkinson disease 10 (PARK10), Parkinson disease 11 (PARK11), Parkinson disease 12 (PARK12), Parkinson disease 13 (PARK13), or Mitochondrial Parkinson's disease. For example, the neurodegenerative disease or disorder comprises Alzheimer's disease, Huntington's disease, Parkinson's disease, multiple system atrophy, and progressive supranuclear palsy.

In embodiments, the term “treating” or “to treat” can refer to clinical intervention in an attempt to alter the natural course of the individual or subject being treated. For example, “treating a disease” can comprise curing a disease, preventing a disease, reducing the incidence of a disease, or ameliorating symptoms of a disease.

In embodiments, the method comprises administering to a subject a therapeutically effective amount of a botanical extract or a botanical composition.

A “botanical composition” can refer to a composition comprising one or more botanical components. For example, the botanical component, is a botanical extract.

A “botanical extract” can refer to a fresh or processed (e.g. cleaned, frozen, dried, sliced, liquified) part of a single species of plant or a fresh or processed alga or macroscopic fungus. In embodiments, the botanical extract can be isolated from the shoot of the plant. The “shoot” of a plant can refer to the plant stem, together with its appendages, leaves and lateral buds, flowering stems, and flower buds. The new growth from seed germination that grows upwards is a shoot where leaves will develop. Stems, which are an integral component of shoots, provide an axis for buds, fruits, and leaves.

The term “botanical extract” or “plant extract” can refer to a product prepared by separating, by chemical or physical process, one portion of a plant from another. For example, a product prepared by separating, by chemical or physical process, any medicinally active portions of a plant from the inactive or inert components. The botanical extracts prepared according to the invention can be obtained by means of a solvent (i.e., a polar solvent or a non-polar solvent), or under pressure and/or heat.

The term “solvent” can refer to a substance that can be dissolved or dispersed in one or more substances.

In embodiments, the plant extracts can be provided by the use of a polar solvent. The term “polar solvent” can refer to a solvent that comprises dipole moments. For example, a polar solvent can be miscible with water and polar solvents. For example, a polar solvent can comprise chemical species in which the distribution of electrons between covalently bonded atoms is not even. For example, the polarity of solvents can be assessed by measuring any parameter known to those of skill in the art, including dielectric constant, polarity index, and dipole moment (see, e.g., Przybytec (1980) “High Purity Solvent Guide,” Burdick and Jackson Laboratories, Inc.). In embodiments, the polar extracts can comprise any percentage of polar solvent including, but not limited to, for example about 1-10% polar solvent, about 10-20% polar solvent, about 20-30% polar solvent, about 30-40% polar solvent, about 40-50% polar solvent, about 50-60% polar solvent, about 70-80% polar solvent, about 80-90% polar solvent or about 90-100% polar solvent. Examples of polar solvents include but are not limited to ethyl alcohol (ethanol), butyl alcohol (butanol), methanol, water, acetic acid, tetrahydrofuran, N,N-dimethylformamide, dichloromethane, ethyl acetate, acetonitrile, dimethylformamide, dimethyl sulfoxide, acetone, or n-propanol.

In embodiments, the plant extracts can be provided by the use of a non-polar solvent (i.e. non-polar extract). As used herein, “nonpolar” and “non-polar” can be used interchangeably. As used herein, the term “nonpolar solvent” can refer to a solvent comprising molecules that do not have an overall dipole. For example, the solvent comprises molecules comprising bonds between atoms with similar electronegativities (e.g. a carbon-hydrogen bond). For example, the nonpolar molecule comprises equal sharing of electrons between atoms or the arrangement of polar bonds leads to overall no net molecular dipole moment. The non-polar extracts of the invention can comprise any percentage of non-polar solvent, including but not limited to, for example, about 1-10% non-polar solvent, about 10-20% non-polar solvent, about 20-30% non-polar solvent, about 30-40% non-polar solvent, about 40-50% non-polar solvent, about 50-60% non-polar solvent, about 70-80% non-polar solvent, about 80-90% non-polar solvent, or about 90-100% non-polar solvent. Examples of non-polar solvents include but are not limited to isooctane, hexane, pentane, benzene, chloroform, diethyl ether, hydrocarbons, cyclohexane, toluene, or 1,4-dioxane.

Hydrophobic molecules can be non-polar and thus can interact with (e.g. associate, aggregate, etc.) other neutral molecules and non-polar solvents. For example, nonpolar or hydrophobic molecules can interact through non-covalent interactions. For example, the non-covalent interaction is a van der Waals interaction. For example, the van der Waals interaction are London forces. Hydrophilic molecules can be polar and dissolve by water and other polar substances.

Thus, the plant extracts can be produced by any method known in the art including a polar extract such as a water (aqueous) extract or an alcohol extract (e.g., butanol, ethanol, methanol, hydroalcoholic, see for example Swanson R L et al., 2004, Biol. Bull. 206: 161-72) or a non-polar extract (e.g., hexane or isooctane, see for example, Ng L K and Hupe M. 2003, J. Chromatogr A. 1011: 213-9; Diwanay S, et al., 2004, J. Ethnopharmacol. 90: 49-55.

Regardless of the exact solvent employed, plant extracts can be made by placing a plant sample (e.g., leaves, seeds, or other part of the plant) in a mortar along with a small quantity of liquid (e.g., 10 ml of water, alcohol or an organic solvent for every 2 grams of plant sample) and grinding the sample thoroughly using a pestle. When the plant sample is completely ground, the plant extract is separated from the ground plant material, such as by centrifugation, filtering, cation-exchange chromatography, and the like, and the collected liquid can be further processed if need be (such as by a concentrating column and the like), active ingredients can be separated from this extract via affinity chromatography, mass chromatography and the like.

The term “aqueous extract” can refer to a plant extract where the extraction has been performed using water as the only solvent. The term “organic extract” can refer to a plant extract where the extraction has been performed using an organic solvent that is not an alcohol. The terms “alcoholic extract” or “alcohol extract” can be used interchangeably. The term “alcoholic extract” can refer to an extract where the extraction has been performed using an alcohol as the solvent. In an embodiment, the extract comprises an alcoholic extract (Ribnicky et al. 2006, Phytomedicine, 13, 550; Longendra et al. 2006, Phytochemistry, 67, 1539; Schmidt et al. 2008, Metabolism, 57, S3; and Ribnicky et al. 2009, Int. J. Pharm., 370, 87). For example, the extract comprises an ethanolic extract. In another embodiment, the extract comprises a polar extract. For example, the extract comprises ethyl acetate. For example, the extract comprises hot water.

As used herein, the term “alcoholic extract” can refer to an extract where the extraction has been performed using a mixture of water and an alcohol, such as methanol or ethanol. For example, the extract is an ethanolic extract. As used herein, the term “ethanolic extract” can refer to an extract where the extraction has been performed using ethanol as the solvent.

In another embodiment, the extract is a dry extract.

For example, the alcoholic extract can be about 10% alcohol extract, about 20% alcohol extract, about 30% alcohol extract, about 40% alcohol extract, about 50% alcohol extract, about 60% alcohol extract, about 70% alcohol extract, about 80% alcohol extract, about 90% alcohol extract, or about 99% alcohol extract.

For example, the alcoholic extract can be about 10%-30% alcohol extract, about 20%-40% alcohol extract, about 30%-50% alcohol extract, about 40%-60% alcohol extract, about 50%-70% alcohol extract, about 60%-80% alcohol extract, about 70%-90% alcohol extract, or about 80%-100% alcohol extract.

For example, the alcoholic extract can be an ethanolic extract. For example, the alcoholic extract is 20% ethanol extract, a 70% ethanol extract, a 100% ethanol extract.

In embodiments, the botanical extract can be a plant extract. As used herein, the term “botanical” can refer to a material that is or can be obtained from a tree, plant-, weed- or herb-derived. As used herein, “botanically derived” can refer to a material that can be derived from a botanical, such as by isolation or extraction; however, “botanically derived” is not limited in this application to materials which actually are isolated or extracted from a botanical, but also includes materials obtained commercially or synthetically.

In embodiments, the botanical extract can be isolated from Artemisia spp. For example, the Artemisia spp. can be Artemisia dracunculus L.

In embodiments, the botanical extract can be an isolated extract. An “isolated extract” can refer to one or more compounds present in or obtained from a plant, such as Artemisia dracunculus L. In an embodiment, the extract can refer to a mixture or blend of compounds present in the plant. Such compound(s) can be obtained by extracting a whole or part of a plant. The extraction step can be optionally followed by further enrichment steps. The terms “extract” and “isolated extract” can be used interchangeably.

In embodiments, the botanical composition can comprise a botanical extract prepared from an entire plant, or a plant extract prepared from a part of a plant, such as flowers, flowering tops, aerial parts, leaves, stems, buds, roots, bulbs, rhizomes, bark, seeds, fruit or fruit peel, bark, kernel, stones, berries, sap, resin, latex and thallus of the plant. In embodiments, the botanical extract is prepared from the plant shoot, such as from the leaves, flowers, and/or stems of a plant. For example, the botanical extract can be prepared form the leaves, flowers, and/or stems of Artemisia spp.

In embodiments, the botanical composition can be prepared by mixing the active plant extracts with one or more inert excipients (e.g., carriers, vehicles, binders, diluents etc.) suitable for the selected route of administration. The term “excipients” can refer to pharmaceutically acceptable organic or inorganic substances which do not deleteriously react with the active compounds. Examples of excipients include, but are not limited to, dietary suitable starch, vegetable oil, vegetable gums, gelatins, soy extracts, sugars, grains, natural and artificial flavorings, and the like. Examples of carriers include, but are not limited to, water, salt solutions, alcohol, plant seed and vegetable oils, glycols, gelatin, lactose, amylose, magnesium stearate, talc, silicic acid, viscous paraffin, perfume oil; fatty acid monoglycerides and diglycerides, fatty acid esters, hydroxymethylcellulose, and the like. The botanical compositions described herein can also be formulated as syrups and elixirs. Further, additional compositions can be readily prepared using technology which is known in the art such as described in detail in Remington's Pharmaceutical Sciences, Twentieth Edition.

The botanical compositions can be in a form suitable for oral use, for example, as tablets, troches, lozenges, pills, aqueous or oily suspensions, solutions, dispersible powders or granules, emulsions, hard or soft capsules, syrups or elixirs, pastes, gels or the like. Compositions intended for oral use can be prepared according to any known method, and such compositions can contain one or more agents selected from the group consisting of sweetening agents, flavoring agents, coloring agents, and preserving agents in order to provide commercially viable, pharmaceutically elegant and palatable compositions. Tablets can contain the active ingredient(s) in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They also can be coated for controlled delivery. For example, a “delayed release” dosage form releases a product or substance at a time other than promptly after administration. Examples of delayed-release systems include repeat-action tablets and capsules, and enteric-coated tablets where timed release is achieved by a barrier coating.

The terms “carrier oils” can refer to any lipid-based carrier materials, oil and/or aqueous solution, derived from any plant seed sources, suitable for administration of the active compounds in the botanical compositions. Carrier oils useful herein include any such materials known in the art that are nontoxic, have stand alone beneficial and therapeutic effects, and do not interact with other components. The term “a pharmaceutically acceptable carrier” can refer to any substantially non-toxic carrier conventionally useable for administration in which the active composition of the invention and its derivatives will remain stable and bioavailable.

Botanical compositions can be formulated as oily suspensions in a pharmaceutically acceptable carrier such as a soft gelatin capsule whereby the active ingredient(s) is (are) mixed with an aqueous solution or oil medium, including suspending the active ingredient in a plant seed derived or vegetable oil, for example hemp seed oil, evening primrose seed oil, borage seed oil, olive oil, sesame oil or coconut oil. The oily suspensions can contain a thickening agent or other agent necessary to produce a commercially viable product, such as, beeswax, paraffin, lecithin, or cetyl alcohol. Sweetening and flavoring agents can be added to provide a palatable oral composition. These compositions can be preserved by the addition of an antioxidant such as ascorbic acid.

Compositions of the invention can be sterilized and/or mixed with auxiliary agents and other excipients including but not limited to other carrier oils, preservatives, glycerins, stabilizers, waxes, wetting agents, emulsifiers, suspending agents, lecithin, esters or partial esters, buffers, coloring agents, flavorings and/or aromatic substances and the like which do not deleteriously react with the active compounds.

In embodiments, the botanical extract comprises PMI5011 or an isolated component thereof. For example, “PMI5011” can refer to the ethanolic botanical extract of Artemisia dracunculus L. For example, the isolated extract comprises less than about 0.01% PMI5011, about 0.01% PMI5011, about 0.05% PMI5011, about 0.10% PMI5011, about 0.15% PMI5011, about 0.20% PMI5011, about 0.25% PMI5011, about 0.30% PMI5011, about 0.40% PMI5011, about 0.50% PMI5011, about 1.0% PMI5011, about 1.5% PMI5011, about 2.0% PMI5011, about 2.5% PMI5011, about 3.0% PMI5011, about 3.5% PMI5011, about 4.0% PMI5011, about 4.5% PMI5011, about 5% PMI5011, about 7.5% PMI5011, about 10% PMI5011, or greater than about 10% PMI5011.

In embodiments, the botanical extract comprises DMC-1, DMC-2, dividigenin, sakuranetin, and/or 6-demethoxycapillarism, or any combination thereof. In embodiments, the botanical extract does not comprise DMC-1 and/or DMC-2.

Embodiments herein can comprise a component isolate from a botanical extract, such as a component isolate from an Artemisia spp. Extract. For example, embodiments herein can comprise a component isolate from an Artemisia dracunculus L. extract. An “isolated component thereof” can refer to any compound or mixture of compounds that is isolated from a plant, a plant extract, or botanical extract. The isolated component can be a single compound, a homologous mixture or blend of similar compounds, or a heterologous mixture of dissimilar compounds. For example, the isolated component comprises an isolated component of PMI5011 thereof. Other non-limiting examples of isolated components comprises DMC-1, DMC-2, davidigenin, sakuranetin, or 6-demethoxycappillarisin. Embodiments can comprise combinations of isolated components, including those described herein.

In embodiments, the isolated extract can comprise a knockout extract (KOE). A “knockout extract” can refer to an extract which contains all components and/or levels of an extract except for at least one target compound. In embodiments, the at least one target compound is deficient. In embodiments, the at least one target compound is completely lacking. A knockout extract can reveal the effects of bioactive compounds in a crude extract. In embodiments, the KOE is deficient at least one of DMC-1 or DMC-2. In some embodiments, the KOE is deficient in DMC-1 and DMC-2. In some embodiments, the KOE is deficient in a combination of components. In embodiments, the KOE completely lacks at least one of DMC-1 or DMC-2. In embodiments, the KOE completely lacks DMC-1 and DMC-2. In some embodiments, the KOE completely lacks a combination of components. See, for example, Yu, Yongmei, et al. “The DESIGNER Approach Helps Decipher the Hypoglycemic Bioactive Principles of Artemisia dracunculus (Russian Tarragon)” Journal of Natural Products (2019), which is incorporated by reference herein in its entirety.

Aspects of the invention are drawn towards therapeutic preparations. As used herein, the term “therapeutic preparation” can refer to any compound or composition that can be used or administered for therapeutic effects. As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions. In embodiments, therapeutic preparations can comprise botanical extracts, knock-out extracts, pharmaceutical compositions, botanical compositions, and any compound used for therapeutic purposes described herein.

In embodiments, the botanical extract can be administered to a subject in a therapeutically effective amount. The term “therapeutically effective amount” can refer to that amount of the therapeutic agent sufficient to realize a desired biological effect, such as treating a disease or disorder, such as a UPS-associated disease.

A “therapeutically effective dose” can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose can refer to combined amounts of the active ingredients that result in the therapeutic effect, whether administered in combination, serially or simultaneously.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition, such as a UPS-associated disease or condition. For example, “treating” a cell proliferation disease, such as cancer, can refer to (or be indicated by) reduced levels of “M” proteins in the blood and urine. The “M” proteins are the abnormal monoclonal antibodies that are produced by the myeloma plasma cells. Plasma cells are derived from antibody-producing B cell lymphocytes; in the case of myeloma plasma cells, there is an overgrowth of the monoclonal antibodies collectively known as “M” proteins. There are several tests that are used to diagnose multiple myeloma, but production of the “M” proteins is central to the diagnosis. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

In an embodiment, the isolated extract modulates the activity of at least one deubiquitinating enzyme (DUB). For example, modulates comprises inhibiting or activating. For example, DUBs comprise USP7, USP14, USp15, UCLH1, UCHL5, and RPN11.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as generally described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, preferably in the form of enteric formulations, e.g. as disclosed in U.S. Pat. No. 5,350,741, for oral administration.

The pharmaceutical or therapeutic carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier will vary widely but will usually be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, with the exception of centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

In embodiments, the botanical extract can be present in a pharmaceutical composition comprising a pharmaceutically acceptable carrier. According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Multi-dose formulations can be prepared as a solution of a compound of the invention in sterile, isotonic saline, stored in capped vials, and if necessary a preservative is added (e.g. benzoates). Fixed dose formulations can be prepared as a solution of the compound in sterile, isotonic saline, stored in glass ampoules, and if necessary filled with an inert gas. Each dose of the compound is stored dry in ampoules or capped vials, if necessary filled with inert gas. The multi-dose formulation demands the highest degree of stability of the compound. When the stability of the compound is low fixed dose formulations can be used. For nasal administration, the preparation can contain a compound of the present invention dissolved or suspended in a liquid carrier, in particular, an aqueous carrier, for aerosol application. The carrier can contain additives such as solubilizing agents, e.g., propylene glycol, surfactants such as bile acid salts or polyoxyethylene higher alcohol ethers, absorption enhancers such as lecithin (phosphatidylcholine) or cyclodextrin, or preservatives such as parabines.

In embodiments, a therapeutically effective amount can comprise a dose of about 0.005 mg/kg to about 1000 mg/kg. In some embodiments, a therapeutically effective amount can comprise a dose of about 0.005 mg/kg to about 10 mg/kg. In some embodiments, a therapeutically effective amount can comprise a dose of about 0.25 mg/kg to about 2 mg/kg. In some embodiments, the therapeutically effective amount is at least about 0.001 mg/kg at least about 0.0025 mg/kg, at least about 0.005 mg/kg, at least about 0.01 mg/kg, at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. However, the skilled artisan will recognize that the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

As described herein, embodiments herein can be formulated into a pharmaceutical composition to be compatible with its intended route of administration. Non-limiting examples of routes of administration include parenteral, e.g., intravenous, intradermal, subcutaneous, oral, inhalation, intranasal, transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Generally, dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In embodiments, administering can comprise the placement of a pharmaceutical composition, such as a composition comprising a botanical extract, into a subject by a method or route which results in at least partial localization of the composition at a desired site such that desired effect is produced.

For example, the pharmaceutical composition can be administered by bolus injection or by infusion. A bolus injection can refer to a route of administration in which a syrine is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration of the botanical extract in a continuous manner, such as to maintain the therapeutic effect (activity) over a prolonged period of time.

In embodiments, the term “subject” or “patient” can refer to any organism to which aspects of the invention can be administered, e.g., for experimental, diagnostic, prophylactic, and/or therapeutic purposes. Typical subjects to which compounds described herein can be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject. The terms “subject”, “individual”, and “patient” can be used interchangeably.

In another embodiment, the extract is administered to a subject orally, intravenously, sub-cutaneously, or transdermally. For example, the subject is administered less than 1 μg of extract, about 1 μg of extract, about 2 μg of extract, about 5 μg of extract, about 10 μg of extract, about 50 μg of extract, about 1 mg of extract, about 5 mg of extract, about 10 mg of extract, about 50 mg of extract, about 100 mg of extract, about 500 mg of extract, about 1 g of extract, or greater than 1 g of extract.

In another embodiment, botanical extracts, botanical compositions, pharmaceutical compositions, and therapeutic compositions described herein can be administered in combination with an additional active ingredient. As used herein, the term “active ingredient” can refer to an ingredient that is biologically and/or pharmaceutically active. For example, the additional active ingredients can comprise one or more anti-cancer agents, one or more anti-inflammatory agents, or one or more neuroprotective agents. For example, the one or more anti-cancer agents can comprise Marizomib, Ixazomib, Borezomib, or Carfilzomib.

Methods for Drug Screening

Aspects of the invention are drawn to a drug screening method. In embodiments, the drug screening method can identify compounds that inhibit or promote the activity of proteins identified involved in disease processes, such as proteins associated with the UPS. For example, the method can be used to identify compounds that can modulate deubiquitylases. For example, such compounds that can modulate deubiquitylases can be used in treatment of diseases or conditions characterized by aberrations in the ubiquitin proteasome system (UPS). For example, such diseases or conditions comprise neurodegenerative diseases, inflammatory diseases and or cell-proliferative diseases (e.g., cancer, such as multiple myeloma).

In embodiments, the drug screening method can be used with a high-throughput screening library to identify new therapeutic compounds, such as those that can be used for the treatment of diseases or conditions characterized by aberrations in the UPS.

For example, a drug screening method comprises a combination of time- and concentration-depended experiments using lysates from different cell lines which are tested with different concentrations of a drug, as well as individual components of the drug using a DUB reporter. For example, the cell line is of multiple myeloma and comprises OPM2 and MM.1S. For example, the drug is an extract, such as an extract as described herein. For example, the extract is a PMI5011 extract. For example, the DUB reporter is Z-LRGG-AMC (SEQ ID NO: 1). Not wishing to be bound by theory, a droplet microfluidic trapping array is used to screen intact cells using a cell permeable DUB reporter.

Aspects of the invention are drawn towards a method of probing enzymatic activity of deubiquitinase enzymes, comprising treating a substance thought to contain deubiquitinase enzymes with an extract isolated from Artemisia dracunculus L.

Kits

Aspects of the invention are directed towards kits for treating disease characterized by aberrations in the ubiquitin proteasome system (UPS), such as a cell proliferation disease. The term “kit” can refer to a set of articles that facilitates the process, method, assay, analysis, or manipulation of a sample. The kit can include instructions for using the kid (eg, instructions for the method of the present invention), materials, solutions, components, reagents, chemicals, or enzymes required for the method, and other optional components.

The botanical extract can be provided in a kit. In one embodiment, the kit includes (a) a container that contains a composition that includes a botanical extract or components thereof, and optionally (b) informational material. The informational material can be descriptive, instructional, marketing or other material that relates to the methods described herein and/or the use of the agents for therapeutic benefit. In an embodiment, the kit also includes a second agent, such as a second agent for treating a UPS-associated disease or disorder. For example, the kit includes a first container that contains the botanical extract or composition comprising the same, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the botanical extract, e.g., in a suitable dose, dosage form, or mode of administration (e.g., a dose, dosage form, or mode of administration described herein), to treat a subject who has a nerve disconnectivity disorder). The information can be provided in a variety of formats, include printed text, computer readable material, video recording, or audio recording, or information that provides a link or address to substantive material.

In addition to the botanical extract, the composition in the kit can include other ingredients, such as a solvent or buffer, a stabilizer, additional active ingredients, or a preservative. For example, the kit can include one or more anti-cancer agents. For example, the kit can include Marizomib, Ixazomib, Borezomib, and/or Carfilzomib. The botanical extract or components thereof can be provided in any form, e.g., liquid, dried or lyophilized form, and can be substantially pure and/or sterile. When the botanical extract is are provided in a liquid solution, the liquid solution can be an aqueous solution or an alcohol solution. When the botanical extract or components thereof are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes the botanical extract and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

A number of studies in vitro and in rodent models of obesity and insulin resistance show that an ethanolic extract from Russian tarragon, termed PMI5011 enhances insulin signaling in skeletal muscle. It is also known that insulin signaling regulates protein degradation via a number of mechanisms, including inhibition of proteasome activity. Our experiments also show that PMI5011 inhibits proteasome activity, but the mechanism is unknown. The deubiquitinase enzymes (aliases are DuB, USPS) are proteases that are reported to affect proteasome activity as well as other functions of the ubiquitin-proteasome system. Using C2C12 myotubes as an in vitro model of skeletal muscle, we isolated whole cell lysate from myotubes that were treated overnight with a solvent vehicle along (DMSO) or PMI5011. We found that PMI5011 consistently inhibits DuB activity in our biochemical assay. Without wishing to be bound by theory, this is a new mechanism of action of PMI5011 that identifies new and specific DuB inhibitors. Our discovery will also lead to new therapeutic tools in the treatment of muscle loss related to insulin resistance.

### Example 2

Aberrations in the ubiquitin proteasome system (UPS) contribute to the pathological states of several clinical disorders, such as cancer, thus indicating that targeting proteolytic and regulatory components of the UPS is an efficient strategy for cancer treatment. While inhibition of the proteasome has proven to be effective in the treatment of multiple myeloma (MM), a significant challenge with these proteasome-targeted therapeutics is the heterogeneity associated with cancer cells. This has led to the development of inhibitors for other enzymes associated with the UPS such as the deubiquitinating enzyme (DUBs). While recent studies have suggested that controlled positive regulation of certain DUBs can stabilize autophagy and levels of protein degradation, there are currently no targeted therapeutics that can positively regulate DUB activity. In this work, a botanical extract from Artemisia dracunculus L (herein referred to as PMI5011) was characterized for its effects on DUB activity in two model MM cell line (OPM2 and MM.'S) extracts. Prior studies have been informative on the potential of PMI5011 as a potential therapeutic for diabetes; however, they have not investigated the role of the plant extract on DUB activity in cancer cells. The total extract PMI5011 contains several bioactive components of which five have been identified to exhibit bioactivity: DMC-1, DMC-2, davidigenin, sakuranetin and 6-demethoxycapillarisin. An in-depth enzymology analysis was performed using a commercially available fluorescent DUB reporter to demonstrate a significant effect of PMI5011 and three bioactive components (DMC-1, DMC-2, and davidigenin) on DUB activity. Concentration-dependent studies and mathematical modeling revealed that DUB kinetics in the presence of PMI5011, in both OPM2 and MM.'S lysates, followed a heterotropic co-operativity model where PMI5011 bound with the active domains of DUBs to directly or indirectly enhance their activity without affecting the cellular viability. A similar kinetic profile was observed for DMC-1, DMC-2, and davidigenin; however, their influence on DUB activity was found to be cell line dependent at higher concentrations. Interestingly, davidigenin was found to inhibit DUB activity in MM.1S cells but enhance in OPM2 cells. Whereas, a knock-out extract (KOE) missing both DMC-1 and DMC-2 was observed to enhance DUB activity in both OPM2 and MM.1S lines indicating that these two bioactive components were not the primary compounds to stimulate DUB activity in the PMI5011 plant extract. An mRNA profile revealed that this cell dependent effect of PMI5011 and its components on OPM2 and MM.1S was due to differential expression of specific DUBs such as USP7, USP15, USP14, Rpn11 in OPM2 and MM.'S. These findings demonstrating the complexity of the PMI5011 plant extract and its various bioactive components on DUB regulation reveal their therapeutic properties, but also shine light on the allosteric nature and structural specificity of these enzymes.

### Example 3—Characterization of PMI-5011 on the Regulation of

### Deubiquitinating Enzyme Activity in Multiple Myeloma Cell Extracts

Abstract

Deubiquitinating enzyme (DUB)-targeted therapeutics can be used as alternative cancer therapeutics, especially when coupled with proteasome-based inhibitors. While DUB-based therapeutics function by inhibiting DUB enzymes, positive regulation of these enzymes can stabilize levels of protein degradation. Unfortunately, there are no clinically available therapeutics for this purpose. Work herein is direct towards characterizing the effect of a botanical extract from Artemisia dracunculus L called PMI-5011 on DUB activity in cancer cells. Through a series of kinetic analyses and mathematical modeling, it was found that PMI-5011 positively regulated DUB activity in two model multiple myeloma cells line (OPM2 and MM.1S). This data shows that PMI-5011 can interact with the active domains of DUBs to enhance their activity directly or indirectly, without affecting cellular viability. Similar kinetic profiles of DUB activity were observed with three bioactive compounds in PMI-5011 (DMC-1, DMC-2, davidigenin). Interestingly, a differential cell line-independent trend was observed at higher concentrations which indicates variances in inherent gene expressions of UCHL1, UCHL5, USP7, USP15, USP14, and Rpn11 in OPM2 and MM.1S cell lines. These findings show the therapeutic utility of PMI-5011 and its selected bioactive compounds in cancer.

Introduction

The ubiquitin-proteasome system (UPS) is a well-controlled biochemical pathway for the recognition and degradation of misfolded, damaged or dysregulated proteins.¹ The pathway requires a concerted action of a series of enzymes: E1 ubiquitin activating enzymes, E2 ubiquitin conjugating enzymes and E3 ubiquitin ligases, which are responsible for attaching a polyubiquitin chain to a lysine residue on a target protein marking them for proteasomal degradation.² There are also dozens of deubiquitinating enzymes (DUBs) that can reverse this process by removing the polyubiquitin chain from the target protein to rescue the proteins from degradation.³ Thus, the degradation activity of the UPS calibrates the abundance of intracellular proteins not only for homeostatic regulation, but also to mediate changes in metabolism in response to an external signal or for cell cycle progression. Aberrations in the UPS contribute to the pathological states of several clinical disorders including, but not limited to, inflammation, neurodegeneration, and cancer.⁴ Human cancer cells possess elevated levels of proteasome activity and are more sensitive to proteasome inhibitors than normal cells, thus indicating that targeting proteolytic and regulatory components of the UPS is can be a strategy for cancer treatment. For example, proteasome inhibition has been effective in the treatment of multiple myeloma (MM). Proteasome inhibitors include Marizomib (in phase III trial) and Ixazomib (marketed as Ninlaro). Others, such as, Bortezomib (marketed as Velcade) and Carfilzomib (marketed as Kyprolis), can inhibit the chymotrypsin activity of the 20S proteasomal subunit showing efficacy in enhancing the treatment of MM.⁵⁻⁶ A challenge with proteasome-targeted therapeutics is the heterogeneity associated with cancer cells where some patients respond to selective drugs while others develop resistance leading to relapse and death.⁷ This has led to the development of inhibitors for other enzymes associated with the UPS including DUBs. Tian et al. identified potent inhibitors for three DUBs (USP7, USP14 and UCHL5) that help to overcome Bortezomib resistance and induce apoptosis in MM cells.⁸ An inhibitor of UCH-L5 and USP14 was designed that reduced tumor progression in four different solid tumor models of acute myeloid leukemia.⁹ Similarly, several ubiquitin variant inhibitors of USP15, a DUB that is known to dysregulate SMURF2 and TRIM25 substrates of the transforming growth factor 13 pathway in glioblastoma, were developed and characterized for breast and ovarian cancers.¹⁰

While DUB inhibition has shown efficacy in cancer-targeted therapeutics, controlled positive regulation of certain DUBs can stabilize protein degradation and autophagy. Increased levels of USP8 (also called Ubpy) can regulate self-ubiquitination of Nrdp1, a RING finger containing E3 ubiquitin ligase in C2C12, 293T cells, thus increasing its stability.¹¹ OTUB1 inhibited mTORC1 activity by deubiquitinating and stabilizing the inhibitor DEPTOR in response to amino acid deprivation in 293T cells.¹² Similarly, a loss-of function screen of DUBs in HeLa cells identified USP20 as the first DUB to be involved in regulating ULK1 ubiquitination and stability, which is a serine/threonine protein kinase and is an inducer of autophagy.¹³ While these efforts identified how increased DUB activity regulates protein stability, there are no targeted therapeutics that can positively regulate DUB activity. Development of such a therapeutic can, without wishing to be bound by theory, stabilize autophagy and protein degradation in several diseases like cancer, heart disease, neurodegeneration, diabetes, and aging. We can characterize a botanical extract from Artemisia dracunculus L (herein referred to as PMI-5011) for its regulation (either positive or negative) on DUB activity and to understand its role as a ubiquitin proteasome therapeutic in cancer. Botanical extracts have been an important source of medically beneficial compounds. Green tea polyphenols and the microbial metabolite lactacystin have been shown to be potent proteasome inhibitors in the treatment of several cancers.¹⁴ PMI-5011 was identified from a screen of extracts for hypoglycemic activity in diabetic mice with it demonstrating efficacy as a nutritional supplement for diabetes.¹⁵ PMI-5011 decreased the levels of protein tyrosine phosphatase 1B (PTP1B) resulting in decreased and improved insulin levels in mice¹⁶⁻¹⁷. Moreover, PMI-5011 exhibits antidiabetic properties in mouse myoblast C2C12 cells in addition to differentially regulating the expression of genes encoding a range of enzymes associated with the UPS.¹⁸ PMI-5011 inhibits the chymotrypsin-like and caspase-like proteasome activity and also regulated the expressions of two E3 ubiquitin ligases, Atrogin-1 and MuRF-1.¹⁹ Gene profiling results also revealed that PMI-5011 regulated levels of DUB genes USP14, USP19 in Gastrocnemius and Vastus Lateralis muscle cells.¹⁸

These studies have been informative on the effects of PMI-5011 as a therapeutic for diabetes; however, they have not investigated the role of the plant extract on DUB activity in cancer cells. In this work, a kinetic analysis was performed to identify the effect of PMI-5011 on the regulation of DUB activity in two model MM cell lines (OPM2 and MM.1S). The total extract PMI-5011 contains several bioactive compounds of which five have been identified to exhibit bioactivity: DMC-1, DMC-2, davidigenin (DVG), sakuranetin, and 6-demethoxycapillarisin.²⁰ An enzymology analysis was performed using a commercially available fluorescent DUB reporter to demonstrate a significant effect of PMI-5011 and three selected bioactive compounds (DMC-1, DMC-2, and DVG) on DUB activity. Concentration-dependent studies and mathematical modeling revealed that PMI-5011 and its bioactive compounds alter DUB kinetics in two model multiple myeloma cell lines (OPM2 and MM.1S) either enhancing or inhibiting DUB activity depending on the concentration and the compound. A similar kinetic profile was observed for lysates treated with DMC-1, DMC-2, and DVG; however, their influence on DUB activity was found to be cell line dependent at higher concentrations. Interestingly, DVG was found to inhibit DUB activity in MM.1S cells but enhance it in OPM2 cells. Whereas, a knock-out extract (KOE) missing both DMC-1 and DMC-2 was observed to enhance DUB activity in both OPM2 and MM.1S lines indicating that these two bioactive compounds were not the primary compounds to stimulate DUB activity in the PMI-5011 plant extract. An mRNA profile revealed that this cell line dependent effect of PMI-5011 and its bioactive compounds on OPM2 and MM.1S was due to differential expression of specific DUBs such as USP7, USP15, USP14, and Rpn11 in OPM2 and MM.'S. These findings demonstrate the complexity of the PMI-5011 plant extract and its selected bioactive compounds on DUB regulation and reveal their therapeutic properties.

Materials and Methods

Chemicals and Reagents

All reagents in the syntheses of the selected bioactive compounds of PMI-5011 (DMC-1, DMC-2, and davidigenin) were purchased from Sigma Aldrich (St. Louis, Mo.) and used without further purification. The commercial DUB reporter, Z-LRGG-AMC (Z-Leu-Arg-Gly-Gly-AMC) (SEQ ID NO: 1) was purchased from Boston Biochem (Cambridge, Mass.) while AMC [7-Amino-4-methylcoumarin] and dimethyl sulfoxide (DMSO) were purchased from Sigma Aldrich (St. Louis, Mo.). M-PER (mammalian protein extraction reagent) was purchased from Thermo Fisher Scientific (Carlsbad, Calif.). Direct-zol RNA MiniPrep kit was purchased from ZYMO Research (Irvine, Calif.). Multiscribe Reverse Transcriptase and PowerUP SYBR Green Master Mix were purchased from ThermoFisher (Waltham, Mass.). The BCA protein assay kit was purchased from ThermoFisher. All the salts and other reagents used in this study for the preparation of assay buffers were purchased from Sigma Aldrich (St. Louis, Mo.) unless otherwise noted.

Extraction and Synthesis of PMI-5011, KOE, and Selected Bioactive Compounds

The ethanolic extract of Artemisia dracunculus L, PMI-5011 and its knockout extract, KOE were provided by the Botanical Research Center at Pennington Biomedical Research Center. PMI-5011 was obtained from plants grown hydroponically in greenhouses under uniform and strictly controlled conditions, thereby standardizing the plants for their phytochemical content. Detailed information about quality control, preparation and extraction of PMI-5011 and KOE has been previously reported.^{15-16, 20-24} The selected bioactive compounds of PMI-5011 (DMC-1, DMC-2, and DVG) were chemically synthesized at the LSU AgCenter School of Plant, Environmental and Soil Sciences as described herein using modified procedures previously reported.²⁵

Cell Culture and Lysate Generation

OPM2 cells were maintained in RPMI 1640 media supplemented with 12% FBS, 21.8 mM glucose, 8.6 mM HEPES (pH 7.4) and 1.0 mM sodium pyruvate. MM.1S cell line were maintained in RPMI 1640 media supplemented with 10% FBS. The cells were all cultured in T175 flasks (VWR, Radnor, Pa.). All media components were from Corning, Atlanta, Ga. unless otherwise noted. To test the effect of the ethanolic extract and bioactive compounds on DUB activity, cells were exposed to a 16-h pretreatment with 10 μg/mL of PMI-5011 and 1, 10, 30 μg/mL concentrations of DMC-1, DMC-2, KOE, and davidigenin before lysing the cells for experimentation. All compounds were reconstituted in DMSO to obtain stock concentrations, which was further diluted in culture media to obtain the desired experimental concentrations. For vehicle control, a subpopulation of cells was treated with pure DMSO diluted in a similar manner for the same duration. Upon treatment, cell lysates (both OPM2 and MM.1S) were generated by harvesting 1×10⁶ cells/mL, followed by washing 2× and pelleting in phosphate buffered saline (PBS; 137 mM NaCl, 10 mM Na₂HPO₄, 27 mM KCL, and 1.75 mM KH₂PO₄ at pH 7.4). The cell pellet was re-suspended in an approximately equivalent volume of M-PER to the volume of the cell pellet (1000-2000 μL) then vortexed for 10 min at room temperature. Following this, the mixture was centrifuged at 14,000×g for 15 min at 4° C. and the supernatant transferred to a centrifuge tube and stored on ice until use. Total protein concentration was determined using a NanoDrop 2000c (Thermo Scientific, Madison, Wis.).

Murine C2C12 (ATCC; #CRL-1771) were cultured in Dulbecco's modified Eagle's medium (DMEM), high glucose (25 mM) with 10% fetal bovine serum, 2 mM glutamine, and antibiotics (100 units/mL penicillin G and 100 μg/mL streptomycin), in a humidified chamber at 37° C. and 5% CO₂. To obtain fully differentiated myotubes, the medium was exchanged for DMEM, high glucose with 2% horse serum, glutamine, and antibiotics when the myoblasts reached 100% confluence. Thereafter, the medium was replaced every 48 h, and the cells were maintained in this medium until fully differentiated at day 4 post-induction, when the medium was exchanged for DMEM, low glucose (5 mM) with 2% horse serum. The differentiated myotubes were treated with 10 μg/ml PMI-5011 or an equal volume of DMSO overnight in DMEM, low glucose (5 mM) with 0.3% BSA. The adherent myotubes were washed twice with phosphate buffer saline, pH 7.4 (PBS) at 4° C., followed by lysing in M-PER buffer (ThermoFisher, #78505) with 1 mM PMSF. The lysates were sonicated, followed by centrifugation at 14,000×g for 10 min at 4° C. The supernatant was collected, and the protein concentration determined by BCA assay (ThermoFisher). M-PER buffer was used to adjust the concentration to 10 μg/μL and the lysates were stored on ice until used in assays.

Dose-Dependent Treatment and Cellular Viability of PMI-5011 and Selected Bioactive Compounds

The effect of PMI-5011 and bioactive compounds on the viability of cells was tested using a standard colorimetric MTT assay. The MTT Cell Proliferation Assay Kit was purchased from VWR, Radnor, Pa. MM.1S and OPM2 cells were treated with 10 μg/mL of PMI-5011 and 1, 10, and 30 μg/mL of DMC-1, DMC-2, KOE and davidigenin and plated at a density of 106 cells/mL in a 96-well plate with each well containing 250 μL of the sample. The plated cells were then incubated at 37° C. for 16 h. 10 μg/mL for PMI-5011 and a 16-h treatment period was used in this study based on previous findings by Yu et al.²⁴ Parallel plates were prepared for positive and negative solvent vehicle control with the same volume of DMSO and ethanol as for the treated cells. On the day of the experiment, the 96-well plates were centrifuged at 1000×g at 4° C. for 5 min in a microplate compatible centrifuge. The media was discarded and 50 μL of serum-free media and 50 μL of the MTT reagent were added into each well. For a background control, 50 μL of the MTT reagent was added into a well containing media only (no cells). The plate was incubated at 37° C. for 3 h in the dark. After incubation, 150 μL of the MTT solvent was added into each well. The plate was wrapped in a foil and placed on an orbital shaker for 15 min. Finally, the absorbance was measured at λₑₓ=544 nm and λₑₘ=590 nm using a Wallac 1420 VICTOR2 multilabel HTS counter fluorometry (Perkin Elmer (Waltham, Mass.). The percentage viable cells was obtained by Eq. 1.

% Viable Cells=S-N/C-N×100 (Eq. 1)

Where, S denotes raw absorbance values from samples, N denotes average absorbance value of the background control and C denotes the highest average absorbance value of positive vehicle control. The obtained percentage cytotoxicity values were then statistically analyzed for their significance. Each data point represents triplicate data points from duplicate experiments. The effect of PMI-5011 and the selected bioactive compounds on C2C12 cell viability has previously been reported.²⁴

Analysis of Enzyme-Substrate Kinetics in Cell Lysates

The commercial DUB reporter, Z-LRGG-AMC (substrate) (SEQ ID NO: 1) was reconstituted in DMSO to obtain a final stock concentration of 1.44 mM. This stock reporter was diluted as needed in assay buffer (50 mM HEPES, pH 7.4, 150 mM NaCl, 2 mM DTT, and 0.02% Tween-20) supplemented with 4 mg/mL cell lysates (source of DUBs) to reach the desired reporter concentration with a final volume of 100 μL in a 96-well plate. OPM2 and MM.1S cell lysates from different treatments (10 μg/mL of PMI-5011; 1, 10, and 30 μg/mL of DMC-1, DMC-2, KOE, davidigenin and control vehicle DMSO) and C2C12 cell lysates were used to interrogate the effect of the ethanolic extract and selected bioactive compounds on DUB activity. Reporter only and lysates only (noise/background) samples were included in the 96-well plate to confirm that the rates observed were due to DUB-mediated hydrolysis initiated by the cell lysates. The 96-well plate was maintained at a temperature of 30° C. in the dark for the duration of the experiment. The fluorescence signals emitted as a result of DUB-mediated cleavage of AMC (λₑₓ=355 nm and λₑₘ=460 nm) were quantified using a Wallac 1420 VICTOR2 multilabel HTS counter fluorometry (Perkin Elmer (Waltham, Mass.). Readings were collected every 30 min for 6 h. The normalized signals were obtained by comparing the treatment with noise and background fluorescence values which were then statistically analyzed for their significance. Each data point represents triplicate data points from duplicate experiments. A calibration curve was generated for known concentrations of AMC to correlate the fluorescence signal (AU) to concentration (μM). The approximate concentration of free fluorophore was used to calculate reaction rates (μM/min) for each substrate concentration using linear regression analysis.

Statistical Analysis and Numerical Modeling

All data visualization, interpretation and curve-fittings were performed using Origin Pro (OriginLab, Northampton, MA) while statistical analyses of experimental data were carried out using SAS 9.4 (SAS Solutions). The analyses of enzyme-substrate reactions in all cell lysates started with scatter plotting the fluorometry signals (p,M) measured for each substrate concentration against time (min). Fluorescence signals measured for each substrate concentration remained stable during the first 30 min, indicating there was a 30-minute lag period before reaction initiation. Beyond 30 min, the signals demonstrated a linear increase over time as assessed by linear regression and ANOVA statistics. The slopes of each line corresponded to the reaction rate for the given substrate concentrations. In all cases, R2 values above 0.95 were reached for the linear fits. Rates were then plotted against substrate concentrations. Next, non-linear regression analysis was performed to fit the Hill enzymology model (Eq. 2) to the rate data using Levenberg Marquardt iteration algorithm to calculate the kinetic constants Kₘ and Vₘₐₓ.

$$V = \frac{{{V}_{max} [ S ]}^{n}}{{( {K}_{m} )}^{n} + {[ S ]}^{n}} \qquad ( Eq . 2 )$$

where, V denotes reaction rate, Vₘₐₓ denotes maximum reaction rate, [S] denotes substrate concentration, Kₘ denotes half-maximal concentration constant, and n denotes Hill coefficient/co-operativity. Standard Chi-squared tests and ANOVA statistics were used to confirm the goodness of the fit in each case. Based on the non-linear regression analyses, the Hill enzymology model was used over the classic Michaelis-Menten enzymology model due to the multi-layer regulation found in DUB enzymes.²⁶⁻²⁸ The Hill enzymology model can reveal information on co-operativity and allosteric control of these enzymes, wherein the catalytic activity of the enzyme is regulated upon interaction with a molecule. The degree of this interaction is measured by the Hill coefficient which can result in positive co-operativity (n>1) or negative co-operativity (n<1) depending on the concentration, chemical composition and structure of the molecule and the enzyme. The value n=1 denotes no change in catalytic activity resulting in the classic Michaelis-Menten enzymology model.

mRNA Profiling

Total RNA was purified from MM.1S, and OPM2 cell lines using Direct-zol RNA MiniPrep (ZYMO Research, Irvine, Calif.). Total RNA (1000 ng) was reverse transcribed using Multiscribe Reverse Transcriptase (Applied Biosystems, Thermo Fisher Scientific, Waltham, Mass.) with random primers at 37° C. for 2 h. Real-time PCR was performed with PowerUP SYBR Green Master Mix (Applied Biosystems) according to the manufacturer's instructions, using the 7900 Real-Time PCR system and universal cycling conditions (50° C. for 2 min; 95° C. for 10 min; 40 cycles of 95° C. for 15 s and 60° C. for 1 min; followed by 95° C. for 15 s, 60° C. for 15 s, and 95° C. for 15 s). The assays were performed in triplicate, and the results were normalized to HPRT mRNA and analyzed using the 2-ΔΔCT method with USP15 in the MM.1S cells used as the calibrator. The gene and primer sequence list are provided in Table 7.

TABLE 7
Gene and primer sequence list for gene expression analysis.

| Gene ID | Gene Name | Accession # | Sequence-Forward | Sequence-Reverse |
|---|---|---|---|---|
| USP15 | Ubiquitin-specific- | NM_006313 | TCAAAGATGGTGATGC | CTGTTCAACCACCTTT |
| | processing | | CCAGT | CGTG |
| | protease 15 | | SEQ ID NO: 2 | SEQ ID NO: 3 |
| USP7 | Ubiquitin-specific- | NM_003470.3 | TACACCAGGGCGAGCA | GGATGAGACATATTAC |
| | processing | | TTTT | CGGGCT |
| | protease 7 | | SEQ ID NO: 4 | SEQ ID NO: 5 |
| UCHL5 | Ubiquitin C- | NM_001199261 | AGTTTCGCCAGACAGC | GGGTTCCTCTGCAAGT |
| | terminal hydrolase | | AAATG | TGTCT |
| | L5 | | SEQ ID NO: 6 | SEQ ID NO: 7 |
| USP14 | Ubiquitin-specific- | NM_005151.4 | ACCCTCAGCCAAAACT | TCTCAAGGCACCTGCA |
| | processing | | GTCT | TACC |
| | protease 14 | | SEQ ID NO: 8 | SEQ ID NO: 9 |
| Rpn11 | 26S proteasome | NM_005805 | AAACAAGCCATCTATC | GGGTCCTGCTTGCCAA |
| | non-ATPase | | CAGGCA | CATT |
| | regulatory subunit 14 | | SEQ ID NO: 10 | SEQ ID NO: 11 |
| UCHL1 | Ubiquitin C- | NM_004181 | AACCCCGAGATGCTGA | TCTACCCGACATTGGC |
| | terminal hydrolase L1 | | ACAA | CTTC |
| | | | SEQ ID NO: 12 | SEQ ID NO: 13 |
| HPRT | Hypoxanthine | NM_000194 | TGGCGTCGTGATTAGT | GACGTTCAGTCCTGTC |
| | phosphoribosyltran | | GATG | CATAAT |
| | sferase 1 | | SEQ ID NO: 14 | SEQ ID NO: 15 |

Results and Discussion

Synthesis and Characterization of PMI-5011 and Selected Bioactive Compounds

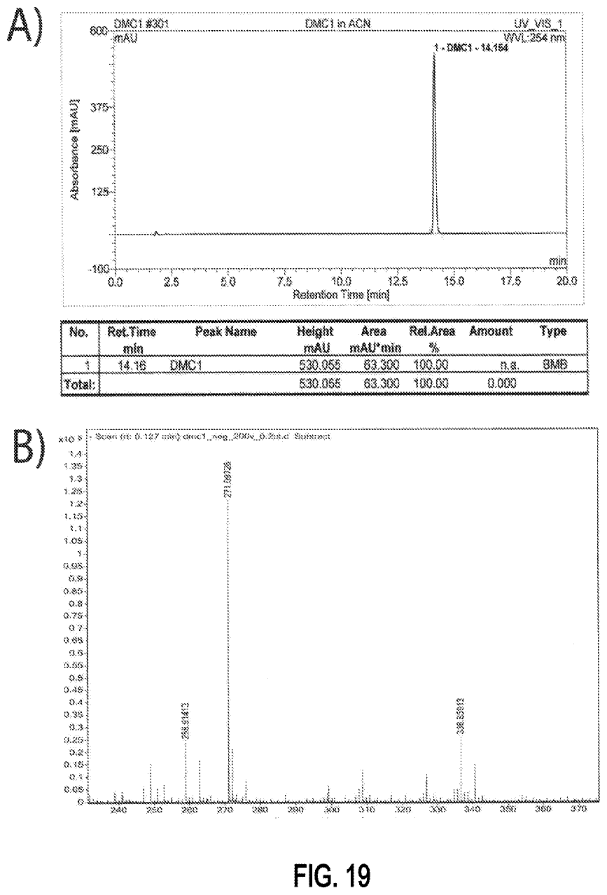

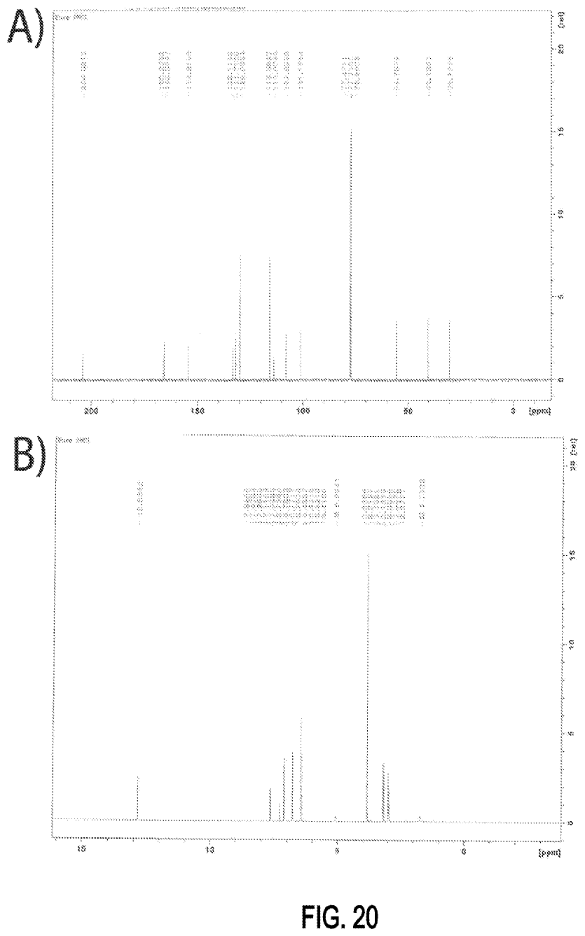

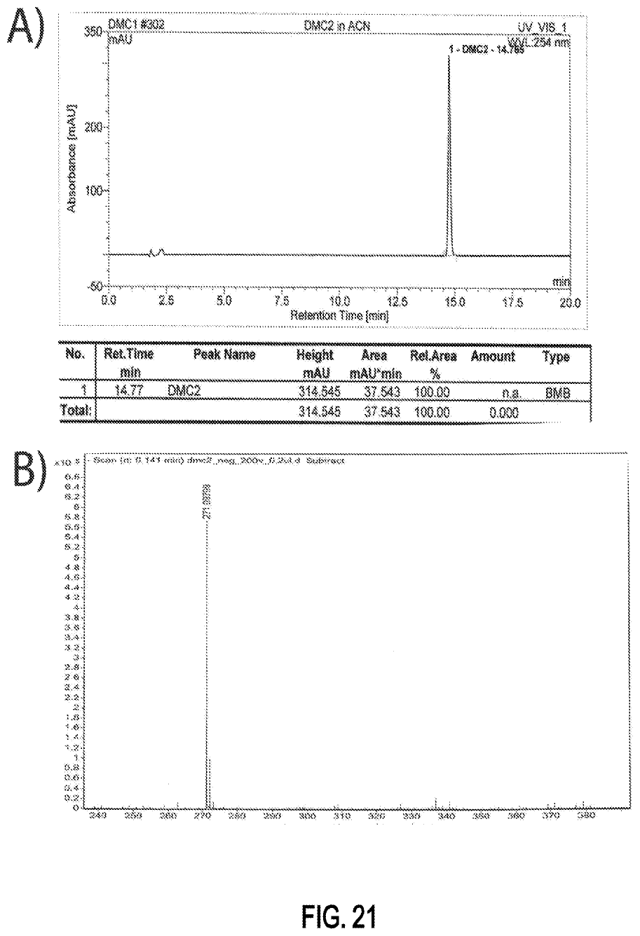

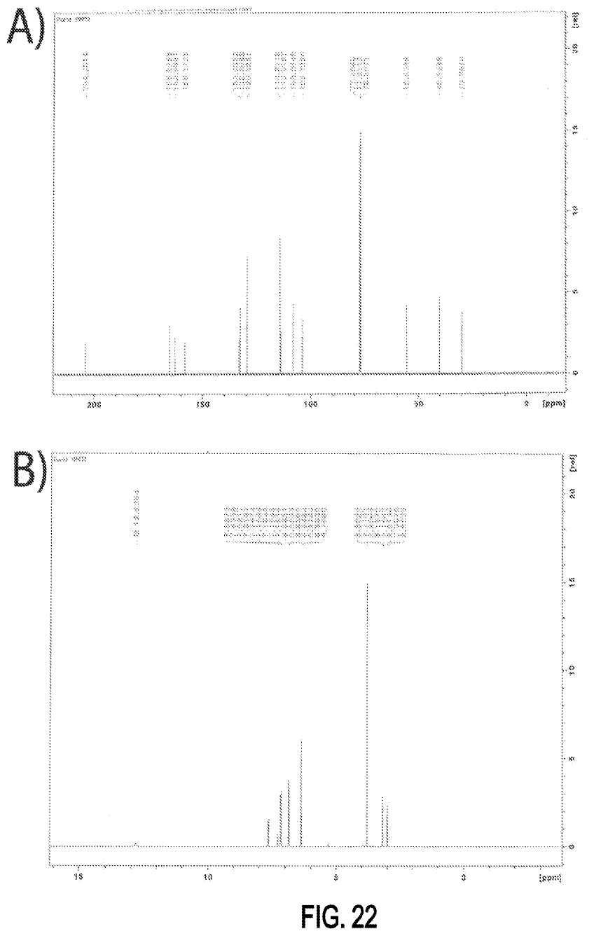

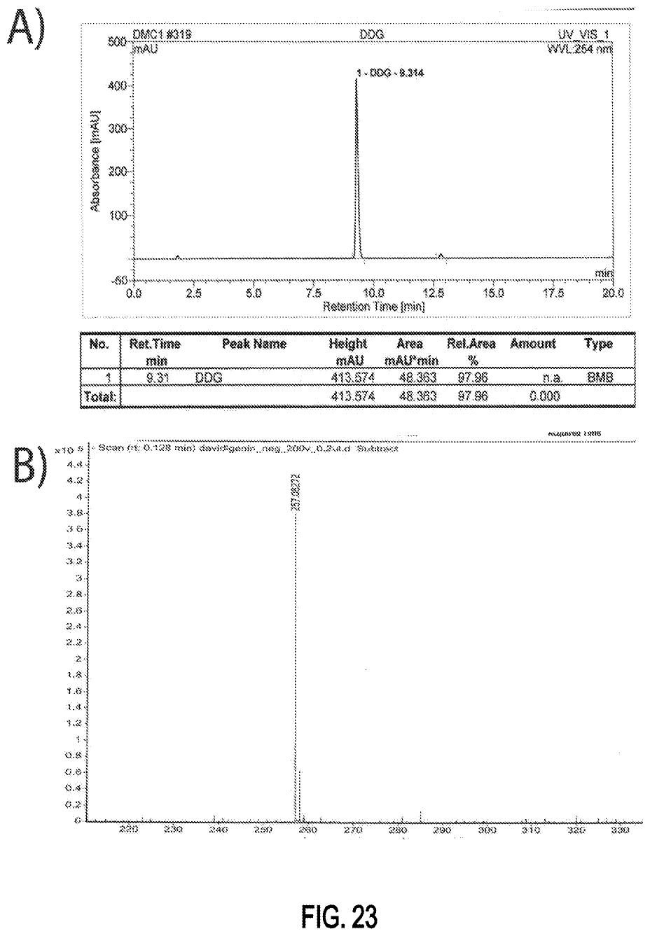

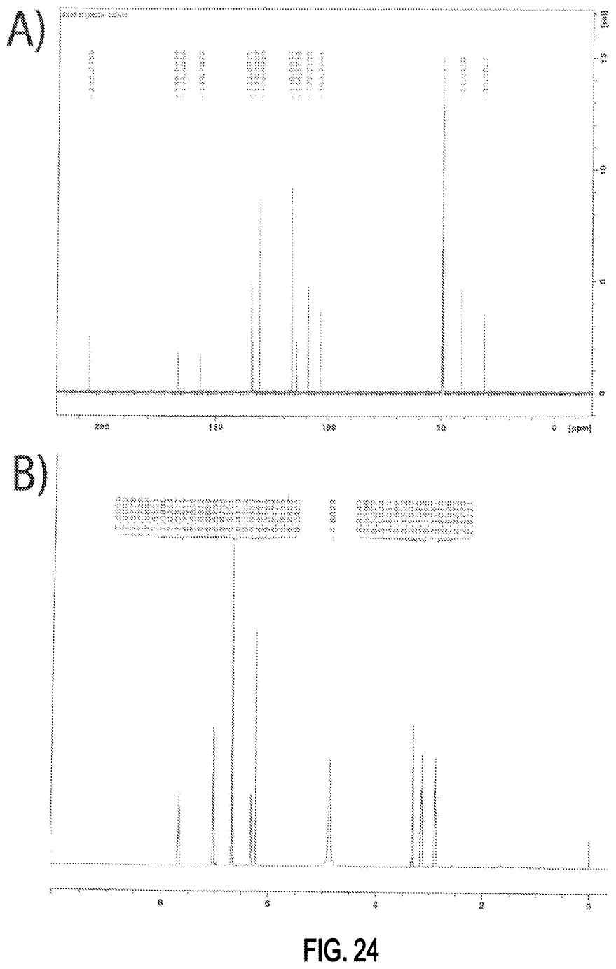

The synthesized DMC-1, DMC-2, and davidigenin (DVG) were characterized in this work and all NMR (¹H and ¹³C) and mass spectrometry characterization data agree with those reported.²⁴ Each compound was synthesized by a base catalyzed aldol condensation reaction with the appropriate ketone and aldehyde under microwave heating. Protection of the various phenolic alcohols as ethoxy methyl ethers were used to prevent any unwanted side reactions. After the aldol condensation reactions were complete, the intermediate olefin was reduced by hydrogenation at atmospheric pressure. Finally, removal of the protection groups was carried out using acid hydrolysis under microwave heating to yield the crude products which were then purified by flash chromatography. Details of each compound synthesis and characterization are presented in the Supplemental Information for DMC-1 (FIGS. 19-20), DMC-2 (FIGS. 21-22), and DVG (FIGS. 23-24) in addition to NMR position data (Tables 3-4).

PMI-5011 Treatment Upregulates DUB Activity in C2C12 and Multiple Myeloma Cells

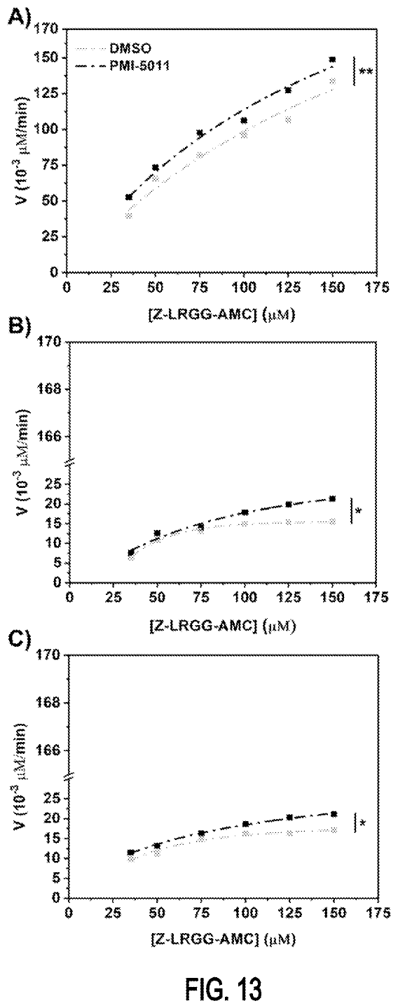

Natural products and their derivatives have been effective for the development of chemotherapeutics displaying structural diversity and pharmacological and molecular characteristics.²⁹⁻³¹ Interestingly, 52% of the total molecules approved from 1981 to 2014 are either natural products or derivatives, out of which some drugs are Paclitaxel (Taxol), Docetaxel (Taxotere), Vincristine (Oncovin) and Vinblastine (Velban) used in breast, testicular, and bladder cancer treatments.³² Herein, a botanical extract from Artemisia dracunculus L (PMI-5011) was characterized for its effect on DUB activity in multiple myeloma cells. There have not been any investigations to understand the role of the plant extract on DUB activity in cancer cells.^{15, 18-19, 33} Here, enzymology studies were carried out to characterize the effect of PMI-5011 on DUB activity using lysates derived from two model multiple myeloma cell lines (MM.1S and OPM2) in addition to C2C12 cells. Duplicate experiments to record the DUB activity revealed that enzyme substrate kinetics followed a Hill enzymology model in all cell lines (FIG. 13). This model was found to be a better fit than the traditional Michaelis-Menten enzymology model as reaction kinetics here involve a multi-layer regulation of DUB enzymes and a complex cellular enzyme-substrate system against pure enzymes. This Hill enzymology model revealed information on co-operativity and allosteric control of these enzymes, wherein the catalytic interaction of the enzyme with molecules (other than substrate) is recorded through Hill coefficient. Table 1 highlights all the kinetic reaction rate constants for control (DMSO) and treatment (PMI-5011) in all three cell lines. From FIG. 13, the DUB activity in C2C12, MM.1S and OPM2 cell lines is enhanced upon PMI-5011 treatment when compared to the DMSO control. Additionally, the inherent DUB activity in C2C12 (FIG. 13 Panel A) upon PMI-5011 treatment was lower when compared with the model multiple myeloma cell lines (FIG. 13 Panel B, Panel C). This is denoted by an unchanged rate constant value Vₘₐₓ (303.25±45.04 μM/min), when compared to the control Vₘₐₓ (310.57±78.21 μM/min). Conversely, for similar substrate concentrations, increased Vₘₐₓ and Kₘ values for OPM2 cells (Vₘₐₓ=36.47±10.87 μM/min, Kₘ=96.86±74.96 μM) and MM.1S cells (Vₘₐₓ=39.78±5.83 μM/min, Kₘ=127.07±33.46 μM), post PMI-5011 treatment correlates with an increase in overall DUB activity.

Besides Vₘₐₓ and Kₘ values, the Hill coefficients obtained through non-linear regression analyses provided some potential insight into the probable interaction between PMI-5011 and DUBs. From Table 1, for C2C12 cells, the unchanged co-operativity (n=0.66±0.37) when compared to the control (n=0.74±0.55) suggest that there might either be lower inherent DUB activity (as previously explained by the unchanged Vₘₐₓ) or no significant interaction between PMI-5011 and DUBs. Whereas for the MM cell lines, an observed decrease in the n values post PMI-5011 treatment (n=1.28±0.53 for MM.1S and n=0.79±0.20 for OPM2) compared against the control values (n=2.94±0.35 for MM.1S and n=1.53±0.53 for OPM2) suggest that the active domains of the DUBs might either be occupied directly by PMI-5011 or other effector molecules that might have been released in its presence to regulate the enzyme activity. Most DUBs of Ubiquitin Specific Protease (USP), Ubiquitin C-Terminal Hydrolase (UCH), Ovarian Tumor Protease (OTU), Machado Joseph Disease Protease (MJD) and Jab1/Mov34/Mpn protease (JAMM) families are multi-domain proteins, consisting of active binding domains and catalytic domains.^{26, 34-35} The obtained reaction rate constants and Hill coefficients indicate that DUB activity in the MM cell lines are regulated in the presence of PMI-5011 and can follow a heterotropic co-operativity model, where a third-party substance (other than enzyme and substrate), PMI-5011, can have either directly or indirectly interacted with the active domain of DUBs to enhance its catalytic activity. The allosteric properties of DUBs describing how specific domains in DUBs like ataxin-3 and UCH-L5 can directly modulate the activity of their catalytic domains have been shown^{27, 36} Also, a linear relationship between ubiquitination and deubiquitination has been indicated.³⁷ Thus, without wishing to be bound by theory, a similar heterotropic relationship can be occurring between PMI-5011 and DUBs here, further shining light on the allosteric properties of DUB enzymes in MM cell lines. While DUB inhibitors can play a role in cancer treatment, results from this work show that PMI-5011 can act as a first-ever positive regulator for DUBs in controlling protein degradation, for example in multiple myeloma. Since PMI-5011 did not have a significant effect on C2C12 cells, further studies were performed only with MM cell lines.

TABLE 1
Reaction parameters of DUB activity for DMSO (control)
and PMI-5011 treated C2C12, MM.1S, and OPM2 cell lines

| | C2C12 | | MM.1S | | OPM-2 | |
|---|---|---|---|---|---|---|
| | Control | PMI-5011 | Control | PMI-5011 | Control | PMI-5011 |
| Vₘₐₓ (10⁻³ | 310.57 ± | 303.25 ± | 15.79 ± | 39.78 ± | 19.02 ± | 36.47 ± |
| μM/min) | 78.21 | 45.04 | 0.36 | 5.83 | 2.10 | 10.87 |
| Kₘ (μM) | 214.78 ± | 166.40 ± | 39.32 ± | 127.07 ± | 35.12 ± | 96.86 ± |
| | 81.14 | 40.25 | 1.12 | 33.46 | 4.60 | 74.96 |
| Vₘₐₓ/Kₘ | 1.45 | 1.822 | 0.40 | 0.31 | 0.54 | 0.38 |
| (min⁻¹) | | | | | | |
| n | 0.74 ± | 0.66 ± | 2.94 ± | 1.28 ± | 1.53 ± | 0.79 ± |
| | 0.55 | 0.37 | 0.35 | 0.53 | 0.53 | 0.20 |

PMI-5011 and Selected Bioactive Compounds Regulate DUB Activity with Minimal Cytotoxic Effects

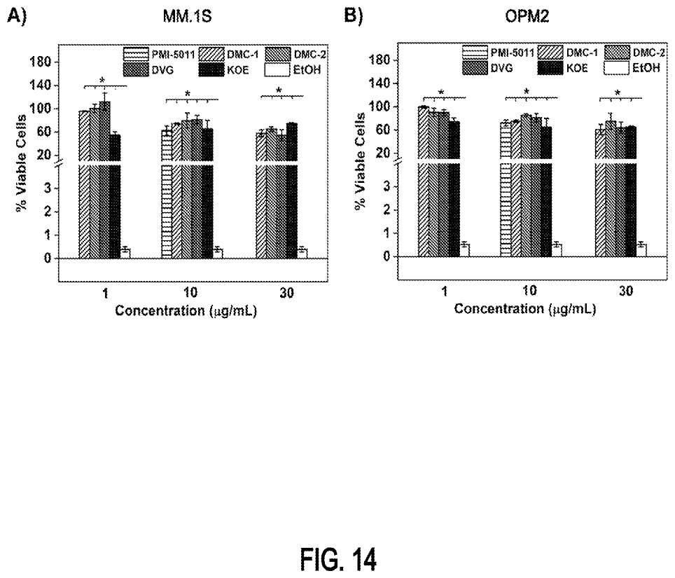

An enzyme or protein regulatory therapeutic can execute its function without affecting the cellular viability. To evaluate the cytotoxicity of PMI-5011 and its selected extracts, dose dependent study was performed on both MM.1S and OPM2 cells using a colorimetric MTT assay. The MTT assay was used due to its compatibility with high-throughput analysis.³⁸ The effect on cellular viability due to therapeutic treatments at 10 μg/mL PMI-5011 and all three concentrations (1, 10 and 30 μg/mL of DMC-1, DMC-2, Davidigenin and KOE) was compared with the negative vehicle control where cells were treated with 70% ethanol. Results from duplicate experiments revealed that MM.1S cells treated with 10 μg/mL of PMI-5011 had a cellular viability of ˜60% compared to the ˜0% cellular viability observed during 70% ethanol treatment (FIG. 14 Panel A). Statistical analysis showed that cellular viability upon compound treatment was significantly greater than that of the negative vehicle control (70% ethanol), indicating that PMI-5011 did not exhibit a strong negative affect on overall cellular viability at a concentration of 10 μg/mL. ANOVA F-statistics was performed to analyze the dose-dependent differences in cellular viability for DMC-1, DMC-2, Davidigenin and KOE. Results from FIG. 14 Panel A and Table 5 show that the percentage of viable MM.1S cells was statistically different for 1 μg/mL and 30 μg/mL treatments of selected bioactive compounds. The average viable cells for the least and highest treatment concentrations were found to be ˜95%±1% and ˜55%±6% for DMC-1, ˜100%±11% and ˜65%±4% for DMC-2, and ˜105%±15% and ˜80%±9% for DVG. For KOE, the percentage of viable MM.1S cells was significantly consistent, ˜50%±6% at 1 μg/mL treatment, ˜65%±14% at 10 μg/mL treatment and a percentage viability of ˜73%±2% at its 30 μg/mL treatment. While cellular viability statistically remained the same at all concentrations of KOE, an inverse linear relationship between cellular viability and treatment concentrations was observed for DMC-1, DMC-2 and Davidigenin. The >100% average cellular viability value observed for 1 μg/mL treatment of Davidignein can be attributed to the nature of the MTT assay. This is because of the raw absorbance value of a sample sometimes being higher than the positive control vehicle, however, the standard deviation of ˜15% in this case (FIG. 14 Panel A) accounts for the lower end of the average cellular viability value. For OPM2 cells, the average percentage of viable cells for the least and highest treatment concentrations were found to be ˜100%±2% and ˜60%±15% for DMC-1, ˜85%±10% and ˜70%±28% for DMC-2, ˜90%±8% and ˜60%±12% for Davidigenin and 75%±8% and 60%±1% for KOE (FIG. 14 Panel B). Statistical analyses (Table 5) revealed that a similar trend was observed in OPM2 cell line, where an inverse relationship was observed between percentage viable cells and increasing treatment concentrations for all the bioactive compounds besides KOE and Davidigenin. Without wishing to be bound by theory, these findings show the features of PMI-5011 and the selective bioactive compounds as therapeutics.

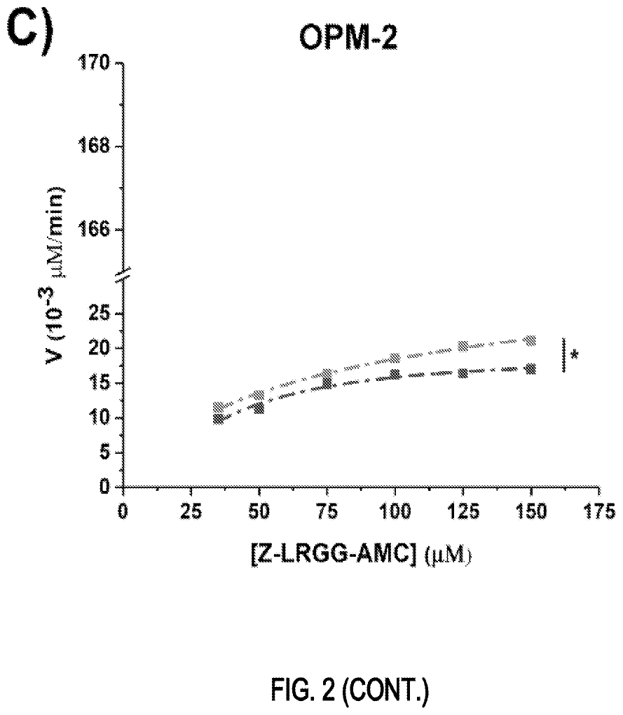

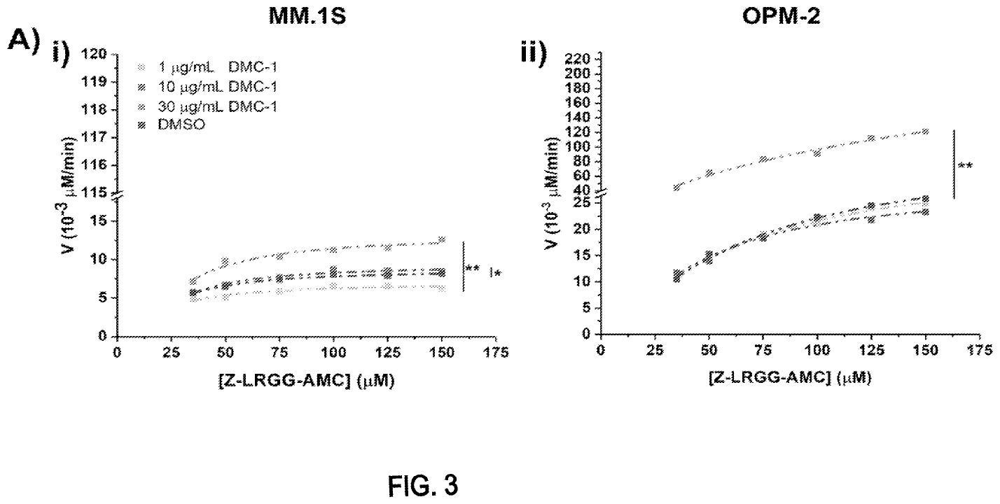

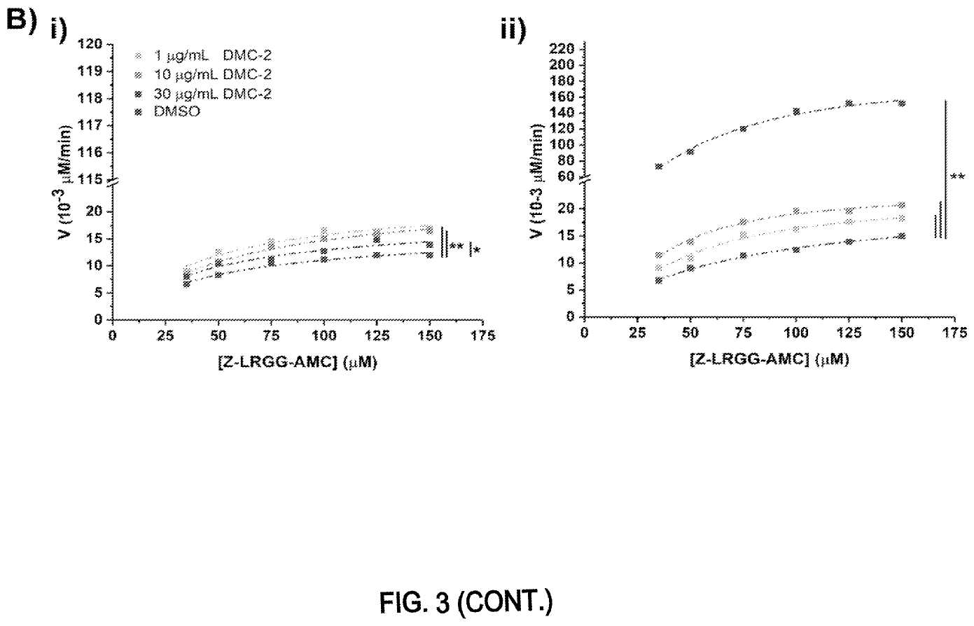

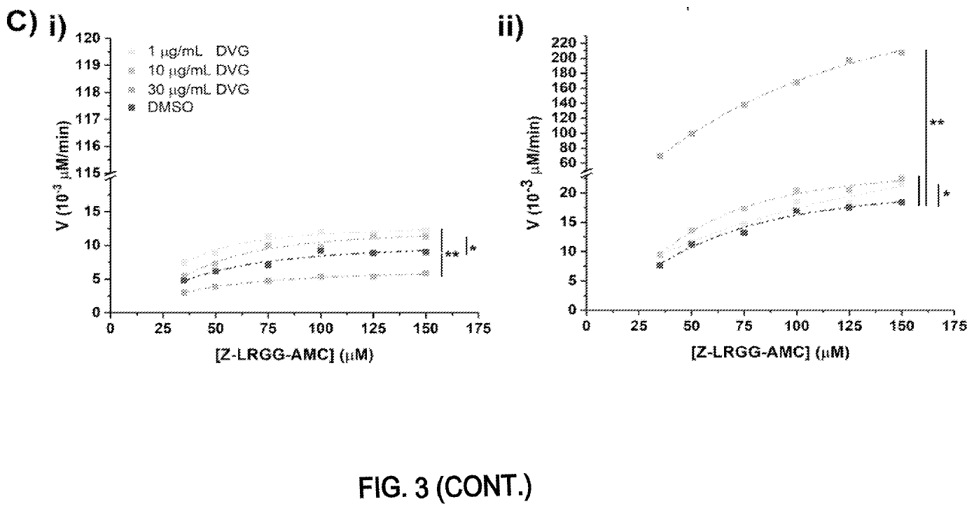

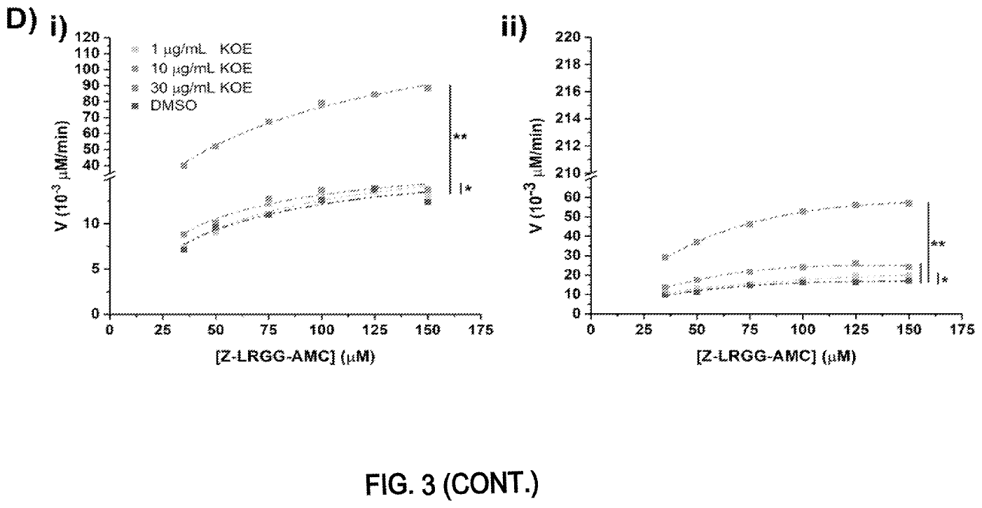

TABLE 5
ANOVA F-statistics analysis on dose-dependent differences
in cellular viability for DMC-1, DMC-2, DVG and KOE.
Null hypotheses: μ_{1 μg/mL} = μ_{30 μg/mL} and μ_{1 μg/mL} =
μ_{10 μg/mL} = μ_{30 μg/mL}. ‘μ’ denotes mean percentage viable cells
in FIG. 2 (e.g., there is no significant change in percentage viable cells
at different treatment concentrations). Alternate: there is a significant
change in percentage viable cells at different treatment concentrations.
p < 0.05 rejects the null in favor of the alternate and is considered as
evidence for a significant change in percentage viable cells at different
treatment concentrations, whereas larger p-values fail to reject the null,
and hence demonstrate an insignificant change in percentage viable cells at
different treatment concentrations. Data demonstrates significant change in
percentage viable cells at 1 ug/mL and 30 treatment concentrations
of DMC-1, DMC-2, DVG but KOE. Davidigenin is denoted as DVG.

| | | |
|---|---|---|
| Null Hypothesis | F(5, 10) | p |

DMC-1

| | | |
|---|---|---|
| μ_{1 μg/mL} = μ_{30 μg/mL} (MM.1S) | 0.25 | <0.05 |
| μ_{1 μg/mL} = μ_{10 μg/mL} = μ_{30 μg/mL} (MM.1S) | 0.24 | <0.05 |
| μ_{1 μg/mL} = μ_{30 μg/mL} (OPM2) | 0.34 | <0.05 |
| μ_{1 μg/mL} = μ_{10 μg/mL} = μ_{30 μg/mL} (OPM2) | 0.41 | <0.05 |

DMC-2

| | | |
|---|---|---|
| μ_{1 μg/mL} = μ_{30 μg/mL} (MM.1S) | 0.32 | <0.05 |
| μ_{1 μg/mL} = μ_{10 μg/mL} = μ_{30 μg/mL} (MM.1S) | 0.48 | <0.05 |
| μ_{1 μg/mL} = μ_{30 μg/mL} (OPM2) | 4.63 | >0.05 |
| μ_{1 μg/mL} = μ_{10 μg/mL} = μ_{30 μg/mL} (OPM2) | 5.84 | >0.05 |

DVG

| | | |
|---|---|---|
| μ_{1 μg/mL} = μ_{30 μg/mL} (MM.1S) | 0.29 | <0.05 |
| μ_{1 μg/mL} = μ_{10 μg/mL} = μ_{30 μg/mL} (MM.1S) | 0.82 | <0.05 |
| μ_{1 μg/mL} = μ_{30 μg/mL} (OPM2) | 3.71 | >0.05 |
| μ_{1 μg/mL} = μ_{10 μg/mL} = μ_{30 μg/mL} (OPM2) | 5.86 | >0.05 |

KOE

| | | |
|---|---|---|
| μ_{1 μg/mL} = μ_{30 μg/mL} (MM.1S) | 10.15 | >0.05 |
| μ_{1 μg/mL} = μ_{10 μg/mL} = μ_{30 μg/mL} (MM.1S) | 6.92 | >0.05 |
| μ_{1 μg/mL} = μ_{30 μg/mL} (OPM2) | 3.23 | >0.05 |
| μ_{1 μg/mL} = μ_{10 μg/mL} = μ_{30 μg/mL} (OPM2) | 8.42 | >0.05 |

Selected Bioactive Compounds of PMI-5011 have Differential Effect on Regulation of DUB Activity in Multiple Myeloma Cell Lines

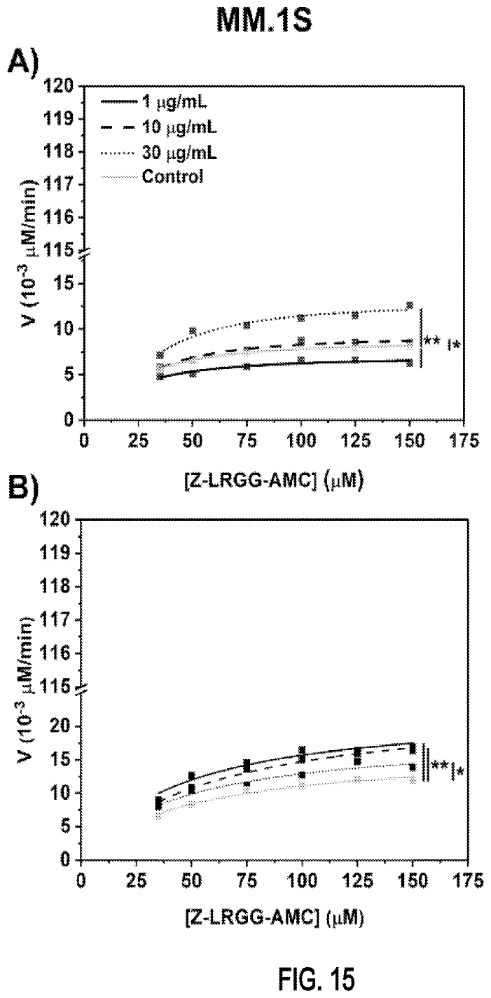

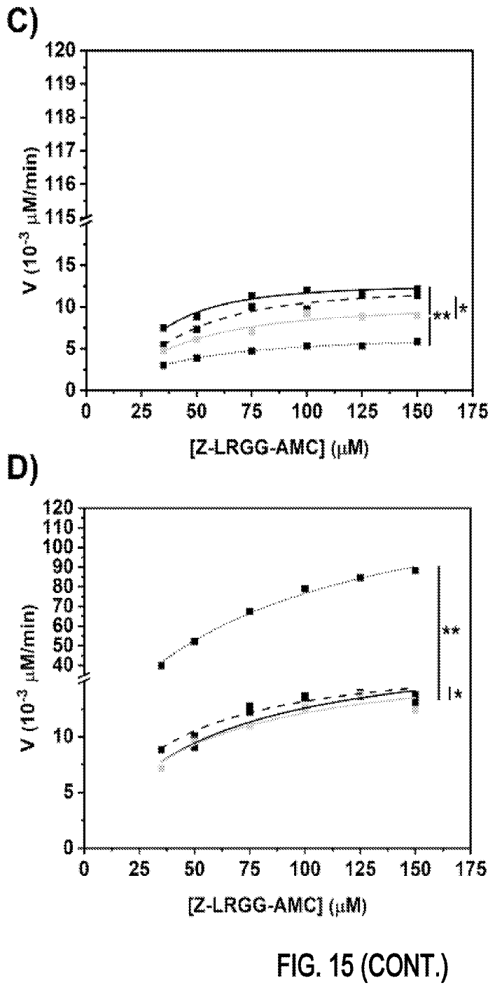

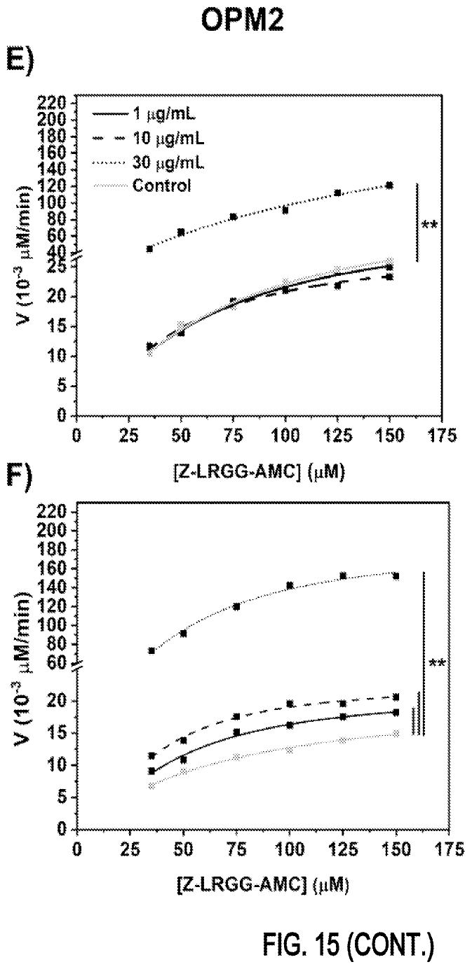

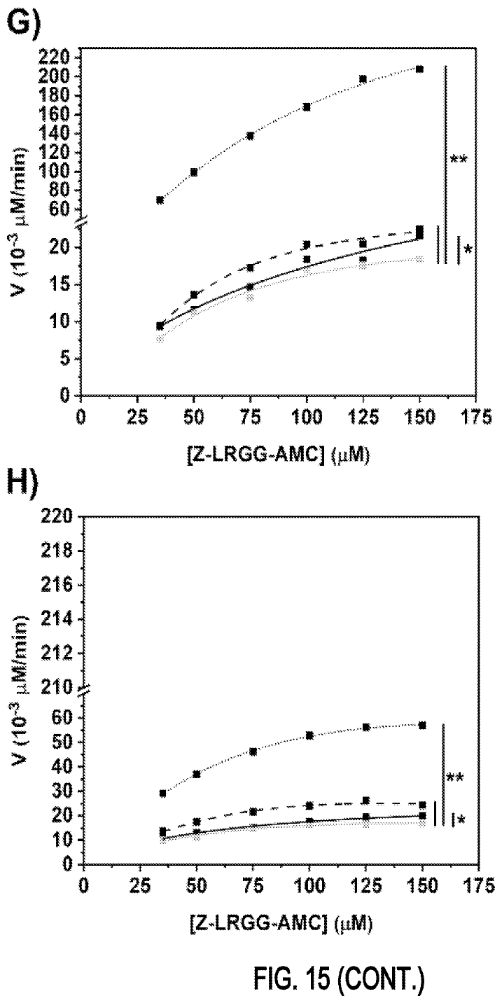

PMI-5011 contains several bioactive compounds including, but not limited to, DMC-1, DMC-2, DVG, sakuranetin, and 6-demethoxycapillarisin. Herein, three of these compounds were synthesized to investigate their effect on DUB activity in cancer cells: DMC-1, DMC-2, and DVG in addition to a knockout extract (KOE) which contains all bioactive compounds except DMC-1 and DMC-2.24 While results from sections herein show that the total plant extract, PMI-5011, interacted with DUB enzyme to enhance its activity, we will determine how its bioactive compounds regulated DUB activity. Both model multiple myeloma cell lines (OPM2 and MM.1S) were exposed to three different doses (1, 10, and 30 μg/mL) of DMC-1, DMC-2, DVG, and KOE (FIG. 15). It can be seen that DMC-1 (FIG. 15 Panel A, Panel E) exhibited a differential effect on DUB regulation in both the MM.1S and OPM2 cell lines, with lower concentrations of 1 and 10 μg/mL significantly inhibiting DUB activity while the highest treatment concentration of 30 μg/mL significantly enhanced DUB activity. This is denoted in Table 2 by the decreased (or unchanged) Vₘₐₓ values for 1 and 10 μg/mL (7.03±1.10 μM/min and 9.19±0.78 μM/min in MM.1S; 33.06±5.08 μM/min and 27.93±4.80 μM/min in OPM2) and an increased Vₘₐₓ value for the 30 μg/mL (12.91±1.45 μM/min in MM.1S and 233.44±27.31 μM/min in OPM2) when compared to the control Vₘₐₓ=8.62±0.46 μM/min in MM.1S and Vₘₐₓ=34.94±6.20 μM/min in OPM2. All three concentrations of DMC-2 significantly enhanced the DUB activity in both MM.1S and OPM2 cell lines (FIG. 15 Panel B, Panel F); however, an opposite trend in concentration dependency was observed between the cell lines. The highest treatment concentration (30 μg/mL) had the least positive regulation on DUB in MM.1S cells, but had the highest positive regulation in OPM2 cells. This is also indicated by the respective reaction rate constants Vₘₐₓ=18.12±7.21 μM/min in MM.'S and Vₘₐₓ=184.79±22.09 μM/min in OPM2 for the 30 μg/mL treatment against the other treatment concentrations 1 and 10 μg/mL (22.70±1.71 μM/min and 18.13±0.14 μM/min in MM.'S; 21.38±2.68 μM/min and 22.71±1.64 μM/min in OPM2) and the DMSO control's Vₘₐₓ=13.11±0.54 μM/min in MM.'S and Vₘₐₓ=20.98±3.59 μM/min in OPM2 as shown in Table 2.

For treatment with DVG (FIG. 15 Panel C, Panel G), again a differential regulation was observed with an inverse relationship between treatment concentrations and DUB activity in MM.1S cells as evidenced by an enhancement in DUB activity that decreased with the increase in DVG treatment concentrations. While the lowest treatment concentrations significantly enhanced the DUB activity (Vₘₐₓ=12.72±0.78 μM/min for 1 μg/mL and Vₘₐₓ=12.23±1.30 μM/min for 10 μg/mL), the highest concentration of 30 μg/mL significantly inhibited the DUB activity (Vₘₐₓ=6.48±0.54 μM/min against the control Vₘₐₓ=10.31±1.96 μM/min). On the contrary, DVG treatment significantly enhanced the DUB activity in OPM2 cells with a linear relationship between treatment concentration and DUB activity (FIG. 15 Panel G). The highest treatment concentration (30 μg/mL) had the highest positive regulation on DUB activity (Vₘₐₓ=315.24±41.65 μM/min) followed by the 10 μg/mL (Vₘₐₓ=24.76±1.84 μM/min) and 1 μg/mL (Vₘₐₓ=23.32±1.58 μM/min) treatments against the control Vₘₐₓ=22.06±3.59 μM/min. All treatment concentrations of KOE enhanced DUB activity (FIG. 15 Panel D, Panel H), with a linear relationship between the treatment concentration and DUB activity for both the cell lines as evidenced by an enhancement in DUB activity that correlated with an increase in KOE treatment concentrations. This is denoted by the respective reaction rate constants for 1, 10 and 30 μg/mL treatment concentrations (Vₘₐₓ=14.18±0.88 μM/min, 14.98±1.12 μM/min and 108.96±5.49 μM/min in MM.1S; 32.49±11.11 μM/min, 27.18±2.34 μM/min and 67.04±3.87 μM/min in OPM2) when compared to the control Vₘₐₓ=12.51±0.47 μM/min in MM.1S and Vₘₐₓ=19.02±2.10 μM/min in OPM2 as shown in Table 2. Overall, the reaction kinetics follow a similar trend between the bioactive compounds and PMI-5011. Without wishing to be bound by theory, this indicates a heterotropic co-operativity model where each of the bioactive compounds can have directly or indirectly regulated the DUB activity in these cell lines by interacting with the active domains to enhance or inhibit its catalytic activity. This can be interpreted from the Hill coefficients obtained upon the non-linear regression analyses as shown in Table 2, where the co-operativity values (n) that are greater than the values obtained for the DMSO control indicating that the active sites of DUB enzyme have either directly interacted with the treatment compounds or any effector molecules to enhance its activity. Similarly, co-operativity values lesser than the ones obtained for the DMSO control indicates that the active sites of DUB enzyme either directly or indirectly interacted with the treatment compound to sterically hinder the catalytic site of the DUB enzyme, thus inhibiting its activity.

TABLE 2
Reaction parameters of DUB activity for control and
DMC-1, DMC-2, DVG, KOE (1, 10, 30 μg/mL) treated
MM.1S and OPM2 cell lines. Davidigenin is denoted as DVG
DMC-1

| | MM.1S | | | | OPM-2 | | | |
|---|---|---|---|---|---|---|---|---|
| | 1 | 10 | 30 | | 1 | 10 | 30 | |
| | μg/mL | μg/mL | μg/mL | Control | μg/mL | μg/mL | μg/mL | Control |
| Vₘₐₓ | 7.03 ± | 9.19 ± | 12.91 ± | 8.62 ± | 33.06± | 27.93 ± | 233.44 ± | 34.94 ± |
| | 1.10 | 0.78 | 1.45 | 0.46 | 5.08 | 4.80 | 27.31 | 6.20 |
| Kₘ | 20.97 ± | 25.97 ± | 29.28 ± | 23.49 ± | 61.42 ± | 46.30 ± | 140.24 ± | 65.35 ± |
| | 5.08 | 3.03 | 3.64 | 2.10 | 16.18 | 12.15 | 28.39 | 19.93 |
| n | 1.33 ± | 1.64 ± | 1.07 ± | 1.56 ± | 1.29 ± | 1.40 ± | 0.93 ± | 1.29 ± |
| | 1.06 | 0.72 | 0.23 | 0.46 | 0.27 | 0.49 | 0.39 | 0.30 |

DMC-2

| | MM.1S | | | | OPM-2 | | | |
|---|---|---|---|---|---|---|---|---|
| | 1 | 10 | 30 | | 1 | 10 | 30 | |
| | μg/mL | μg/mL | μg/mL | Control | μg/mL | μg/mL | μg/mL | Control |
| Vₘₐₓ | 22.70 ± | 18.13 ± | 18.12 ± | 13.11 ± | 21.38 ± | 22.71 ± | 184.79 ± | 20.98 ± |
| | 1.71 | 0.14 | 7.21 | 0.54 | 2.68 | 1.64 | 22.09 | 3.59 |
| Kₘ | 44.76 ± | 39.36 ± | 42.16 ± | 35.29 ± | 44.82 ± | 35.50 ± | 48.16 ± | 67.02 ± |
| | 9.80 | 0.39 | 31.54 | 1.62 | 8.12 | 3.00 | 8.46 | 22.63 |
| n | ~1.00 | 1.68 ± | 1.06 ± | 1.74 ± | 1.47 ± | 1.59 ± | 1.49 ± | 1.09 ± |
| | | 0.04 | 0.79 | 0.25 | 0.40 | 0.37 | 0.36 | 0.22 |

DVG

| | MM.1S | | | | OPM-2 | | | |
|---|---|---|---|---|---|---|---|---|
| | 1 | 10 | 30 | | 1 | 10 | 30 | |
| | μg/mL | μg/mL | μg/mL | Control | μg/mL | μg/mL | μg/mL | Control |
| Vₘₐₓ | 12.72 ± | 12.23 ± | 6.48 ± | 10.31 ± | 23.32 ± | 24.76 ± | 315.24 ± | 22.06 ± |
| | 0.78 | 1.30 | 0.54 | 1.96 | 1.58 | 1.84 | 41.65 | 3.59 |
| Kₘ | 30.17 ± | 39.22 ± | 38.36 ± | 38.98 ± | 56.37 ± | 45.46 ± | 89.35 ± | 51.59 ± |
| | 2.45 | 4.83 | 4.14 | 9.34 | 13.07 | 4.35 | 18.99 | 12.28 |
| n | 2.01 ± | 1.92 ± | 1.51 ± | 1.59 ± | 0.82 ± | 1.78 ± | 1.34 ± | 1.55 ± |
| | 0.67 | 0.68 | 0.34 | 0.85 | 0.48 | 0.33 | 0.16 | 0.48 |

KOE

| | MM.1S | | | | OPM-2 | | | |
|---|---|---|---|---|---|---|---|---|
| | 1 | 10 | 30 | | 1 | 10 | 30 | |
| | μg/mL | μg/mL | μg/mL | Control | μg/mL | μg/mL | μg/mL | Control |
| Vₘₐₓ | 14.18 ± | 14.98 ± | 108.96 ± | 12.51 ± | 32.49 ± | 27.18 ± | 67.04 ± | 19.02 ± |
| | 0.88 | 1.12 | 5.49 | 0.47 | 11.11 | 2.34 | 3.87 | 2.10 |
| Kₘ | 36.08 ± | 29.60 ± | 52.33 ± | 44.14 ± | 82.77 ± | 35.39 ± | 42.53 ± | 35.12 ± |
| (μM) | 2.62 | 2.50 | 4.13 | 12.83 | 71.49 | 3.36 | 3.49 | 4.60 |
| n | 2.32 ± | 1.73 ± | 1.41 ± | 2.73 ± | 0.82 ± | 1.88 ± | 1.46 ± | 1.53 ± |
| | 0.68 | 0.59 | 0.12 | 0.27 | 0.27 | 0.62 | 0.19 | 0.53 |

PMI-5011 and the Selected Bioactive Compounds Exhibit a Concentration- and Cell Line Dependent Effect on DUB Activity

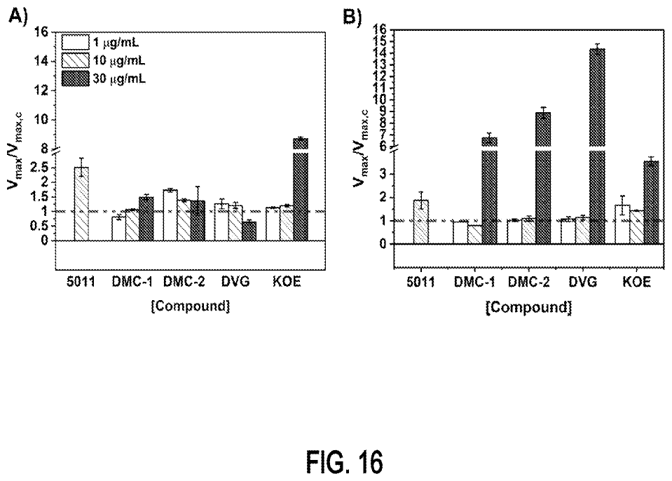

In order to compare and contrast the treatment effects of PMI-5011 and different concentrations of the selected bioactive compounds between the two MM cell lines, the results obtained from the enzyme-substrate reaction kinetics on DUB activity (FIGS. 13 and 15) were compared and contrasted. The maximum reaction rate achieved for the DUB reaction kinetics for all the treatments was normalized against the maximum rate of reaction for the vehicle control DMSO (V_{max,c}) (FIG. 16). Statistical t-tests were performed (FIG. 16 and Table 6) to confirm a significant effect of PMI-5011 and the selected bioactive compounds on DUB activity and to compare this effect between the two MM cell lines. Here, a value greater than one indicates an enhancement of DUB activity, while a value less than one represents an inhibition of DUB activity with the blue dashed line indicating unity based on the normalization. Treatment with 10 μg/mL PMI-5011 was found to significantly enhance DUB activity in both the multiple myeloma cell lines; however, this was observed to have a significantly equal effect in the MM.1S cell line (FIG. 16 Panel A) and the OPM2 cell line (FIG. 16 Panel B). DMC-1 was observed to significantly inhibit DUB activity at lower concentrations (1-10 μg/mL) in both the cell lines, but enhanced DUB activity at higher concentrations (30 μg/mL). An opposite trend was observed in the MM.1S cell line treated with DMC-2 and DVG with an observed decrease in DUB activity for increasing concentrations of the two bioactive compounds. Interestingly, this trend was not observed in OPM2 cells treated with DMC-2 or DVG where an increase in bioactive concentration enhanced DUB activity especially at 30 μg/mL. A similar trend was observed in both cell lines treated with the knock-out extract (KOE) with enhanced DUB activity at the highest dose of KOE (30 μg/mL). Since the KOE contains DVG, but not DMC-1 and DMC-2, these findings highlight the limiting role of DVG in PMI-5011 due to its varied regulation of DUB activity when present in combination with other compounds. KOE was found to enhance DUB activity in both cell lines indicating a role for other bioactive compounds (e.g., sakuranetin, and 6-demethoxycapillarisin). Moreover, the KOE results indicate that DMC-1 and DMC-2 were not the primary compounds to stimulate DUB activity in the PMI-5011 ethanolic extract indicating that sakuranetin, 6-demethoxycapillarisin, or other compounds play a role in modulating DUB activity.

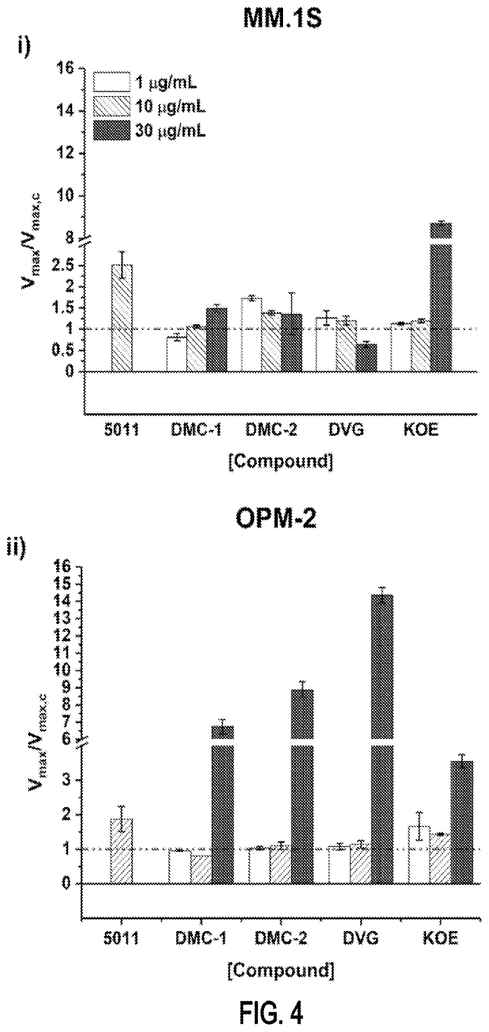

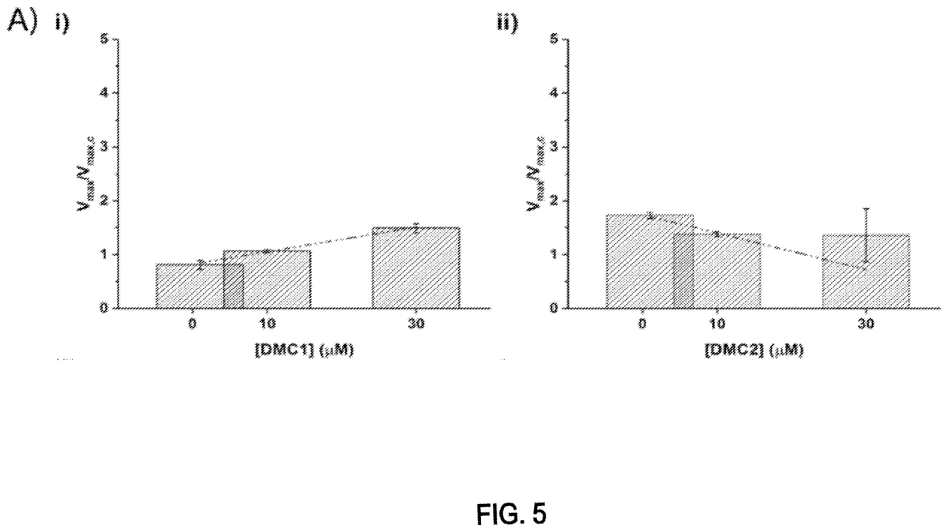

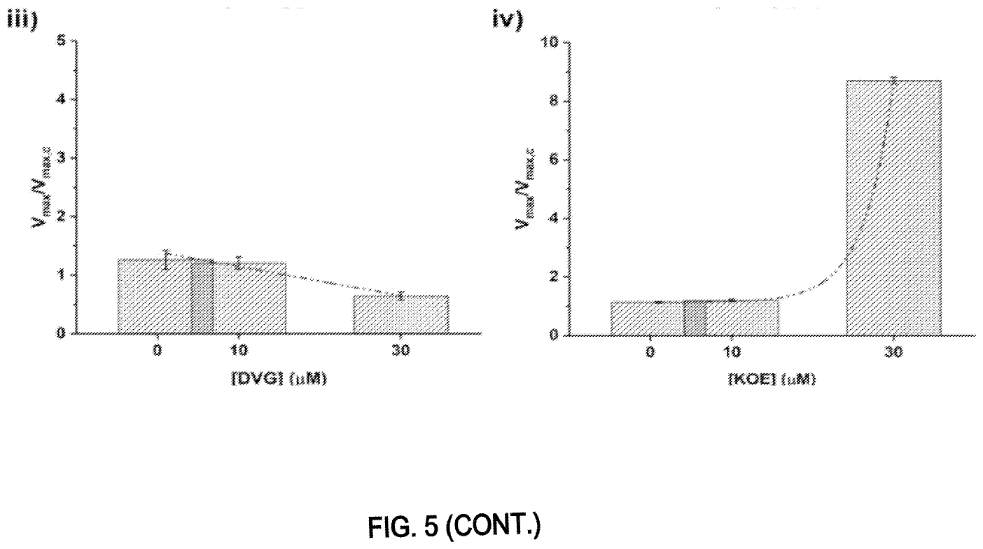

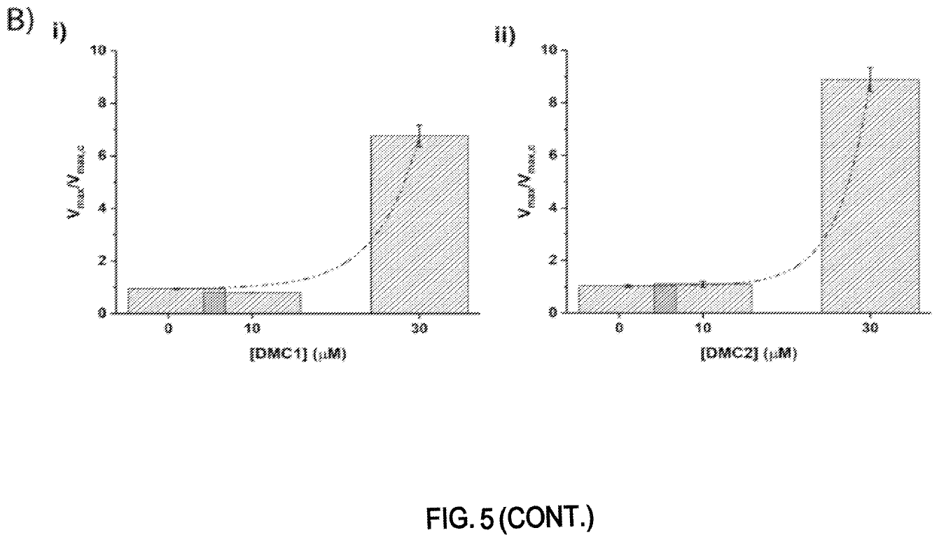

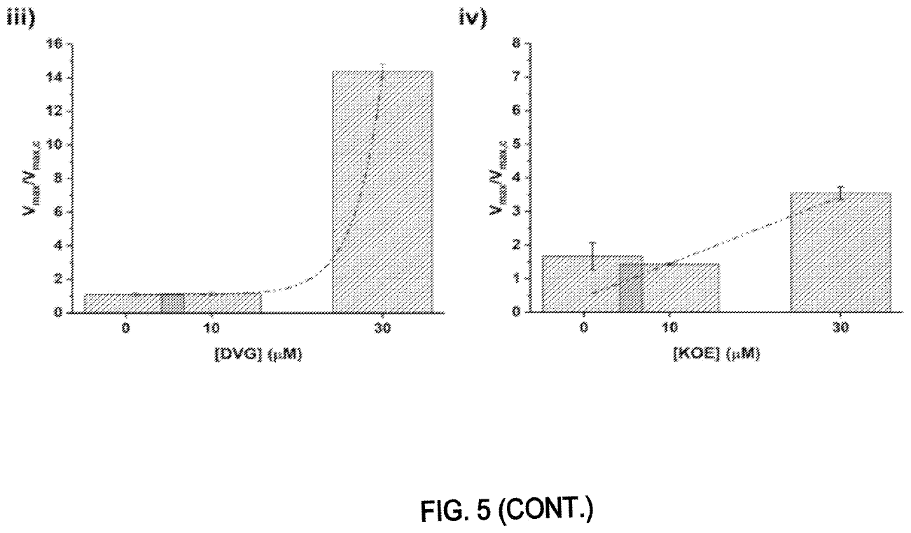

TABLE 6
ANOVA F-statistics analysis on concentration-dependent
effect of the selected bioactive compounds on DUB activity
in MM.1S and OPM2 cell lines. Null hypotheses: μ_{MM.1S} = μ_{OPM2};
μ_{1 μg/mL} = μ_{10 μg/mL} = μ_{30 μg/mL}. ‘μ’ denotes mean normalized reaction rate
as denoted in FIG. 4 (e.g., there is no significant concentration-dependent
effect for the specific treatment in the model cell line). Alternate: there
is a significant concentration-dependent effect for the specific treatment.
p < 0.01 and p < 0.001 reject the null in favor of the alternate and is
considered as evidence for a significant concentration-dependent effect
at different treatment conditions, whereas larger p-values fail to reject
the null, and hence demonstrating no significant concentration-dependent
effect at the specific treatment conditions. Davidigenin is denoted as DVG.

| | | |
|---|---|---|
| Null Hypothesis | F(5, 10) | p |

PMI-5011

| | | |
|---|---|---|
| μ_{MM.1S} = μ_{OPM2} | 5.48 | >0.01 |

DMC-1

| | | |
|---|---|---|
| μ_{1 μg/mL} = μ_{10 μg/mL} = μ_{30 μg/mL} (MM.1S) | 0.24 | <0.01 |
| μ_{1 μg/mL} = μ_{10 μg/mL} = μ_{30 μg/mL} (OPM2) | 0.02 | <0.001 |

DMC-2

| | | |
|---|---|---|
| μ_{1 μg/mL} = μ_{10 μg/mL} = μ_{30 μg/mL} (MM.1S) | 3.48 | >0.01 |
| μ_{1 μg/mL} = μ_{10 μg/mL} = μ_{30 μg/mL} (OPM2) | 0.09 | <0.001 |

DVG

| | | |
|---|---|---|
| μ_{1 μg/mL} = μ_{10 μg/mL} = μ_{30 μg/mL} (MM.1S) | 0.82 | <0.01 |
| μ_{1 μg/mL} = μ_{10 μg/mL} = μ_{30 μg/mL} (OPM2) | 0.02 | <0.001 |

KOE

| | | |
|---|---|---|
| μ_{1 μg/mL} = μ_{10 μg/mL} = μ_{30 μg/mL} (MM.1S) | 0.03 | <0.001 |
| μ_{1 μg/mL} = μ_{10 μg/mL} = μ_{30 μg/mL} (OPM2) | 0.02 | <0.001 |

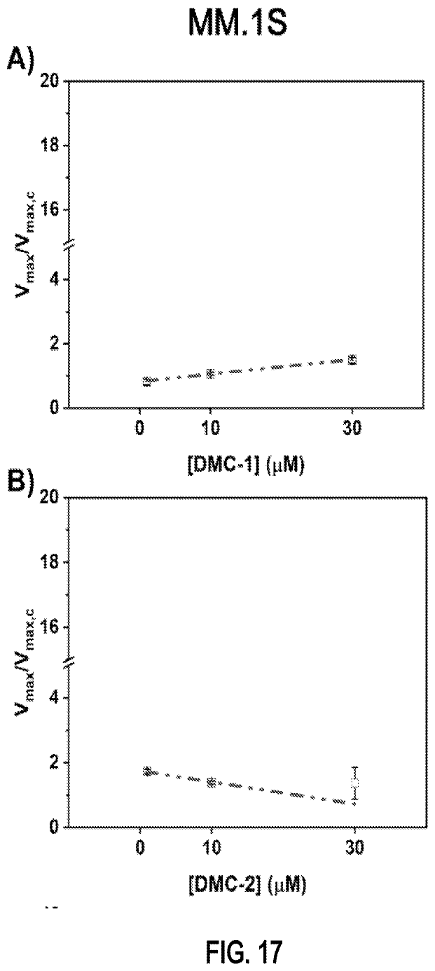

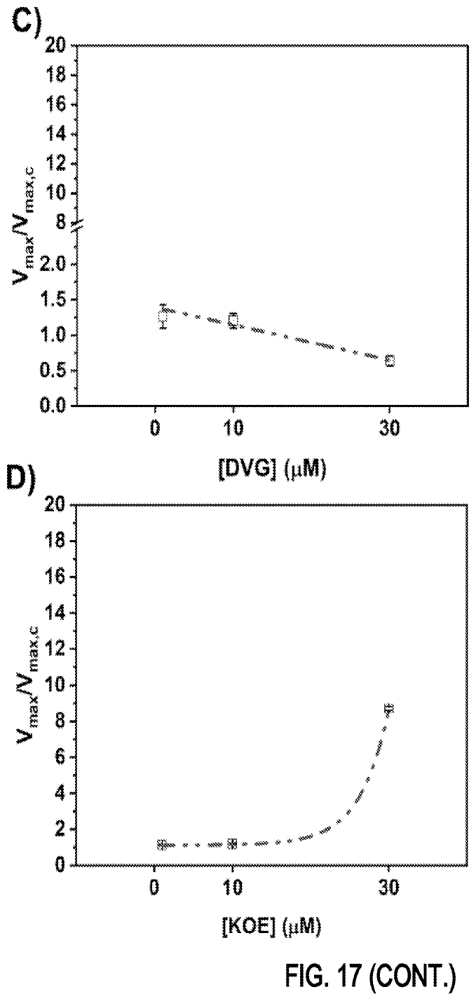

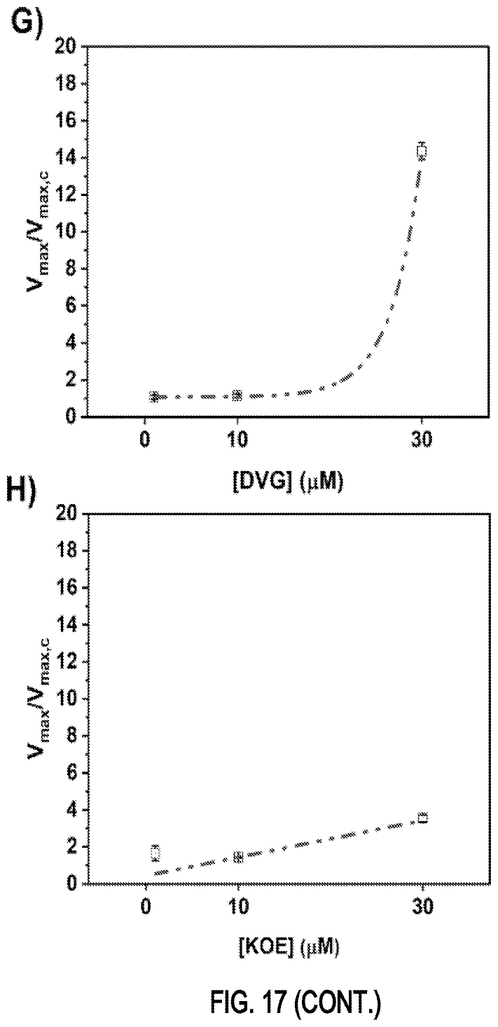

To further interpret the role and effect of bioactive compound concentration on DUB activity in both MM cell lines, the normalized Vₘₐₓ values were plotted against the different compound concentrations (FIG. 17). For MM.1S cells, increasing the concentrations of DMC-1 had a positive linear effect (FIG. 17 Panel A), while DMC-2 and DVG had a negative linear effect on the DUB activity (FIG. 17 Panel B and Panel C). In contrast to this linear effect, an exponential increase in DUB activity was observed with increasing KOE concentrations (FIG. 17 Panel D). An opposite trend was observed in the OPM2 cell line, where a positive linear effect was observed in DUB activity with increasing concentrations of KOE (FIG. 17 Panel H) while there was an exponential increase in DUB activity was observed with increasing DMC-1, DMC-2 and DVG concentrations (FIG. 17 Panels E-G). The difference observed in the linear and exponential trends can provide a biological context for the DUB activity. While a linear increase or decrease in the normalized enzyme-substrate reaction rate with increasing substrate concentrations denotes a constant change in the enzyme-substrate interaction, an exponential increase or decrease in the normalized reaction rate can be due to the exponential change (increase or decrease) in enzyme level occurring because of the specific treatment. The constant decrease in reaction rate from DVG treatment and the exponential increase from the knock-out extract treatment in MM.1S reveals that other bioactive compounds (e.g., sakuranetin, and 6-demethoxycapillarisin) can be responsible for the spike in DUB levels in this cell line. For OPM2, it can be seen that the constant decrease in the reaction rate upon knock-out extract (KOE) treatment but an exponential rate increase in presence of other three selected bioactive compounds confirm that only DMC-1, DMC-2 and DVG are responsible for the increase in enzyme levels. These results can demonstrate information on how the different bioactive compounds derived from PMI-5011 contribute towards regulating DUB activity in OPM2 and MM.1S, despite PMI-5011 in whole exhibiting a similar effect on both these cell lines.

Differential Expression of DUB Gene in MM.1S and OPM2 Contributes to the Cell Dependent Effect of PMI-5011

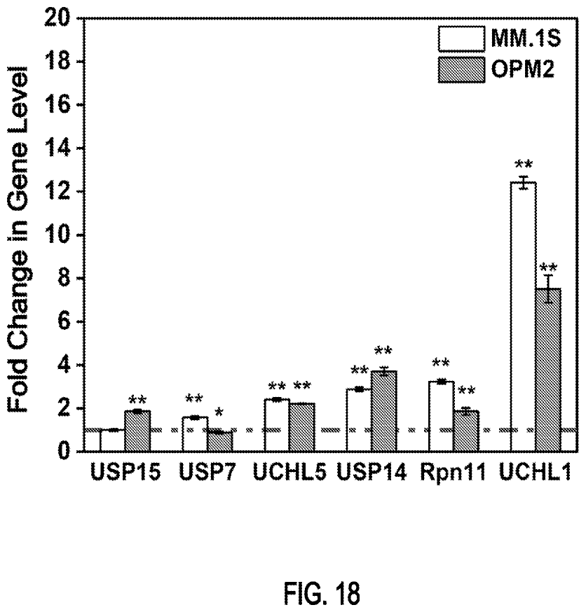

The results herein show that the different bioactive compounds of PMI-5011 interact can differently in regulating DUB activity in the two model MM cell lines, MM.1S and OPM2. One of the reasons for this differential interaction can be due to the inherent differences in these two cell lines, either in gene levels of DUBs or specific post translational modification, thus putting forth a need to provide more biological context to our findings in investigating the reason behind this differential effect. The inherent upregulation of several DUBs such as UCH-L1, UCH-L5, USP7, USP14, PSMD14 (POH1), and CYLD, in both the OPM2 and MM.1S cell lines.^{37, 39-40} Herein, an mRNA profiling experiment was performed to identify specific DUBs potentially targeted by PMI-5011 and the selected bioactive compounds to show if there was any inherent differences in DUB levels in the two cell lines. This can demonstrate information on any inherent differences in the specific DUB gene levels that are commonly unregulated in primary multiple myeloma cancer cells. As shown in FIG. 18, DUB genes were varyingly expressed in both OPM2 and MM.1S cell lines. The results obtained from mRNA profiling were normalized with the least expressed USP15 gene of MM.1S. While UCH-L1 was the most highly expressed gene in both the cell lines, a varying trend was observed with other DUB genes. USP7 was the least expressed in OPM2, followed by Rpn11, USP15, UCH-L5 and USP14. Whereas USP15 was the least expressed in MM.1S followed by USP7, UCH-L5, USP14 and Rpn11. Statistical analyses with p values<0.01 and <0.0001 highlighted that the fold change in gene levels was significantly not equal to that of the control gene. Additionally, p<0.001 values from ANOVA F-statistics (Table 8) to test for any cell-dependent effect clearly reveal that there is a significant difference in DUB gene expressions between MM.1S and OPM2 upon PMI-5011 treatment.

TABLE 8
ANOVA F-statistics analysis on cell-dependent effect of PMI-5011
on DUB activity in MM.1S and OPM2 cell lines. Null
hypotheses: μ_{MM.1S} = μ_{OPM2}; ‘μ’ denotes average DUB gene
expression as denoted in FIG. 18 (e.g., there is no significant difference on
the effect of PMI-5011 between both the cell lines). Alternate: there is a
significant cell-dependent effect of PMI-5011. p < 0.01 and p < 0.001
reject the null in favor of the alternate and is considered as evidence
for a cell-dependent effect on different DUB expressions upon PMI-5011
treatment, whereas larger p-values fail to reject the null, and hence
demonstrating no significant cell-dependent effect. Davidigenin is
denoted as DVG.

| Null Hypothesis | F(5, 10) | p |
|---|---|---|
| USP15, μ_{MM.1S} = μ_{OPM2} | 0.018 | <0.001 |
| USP7, μ_{MM.1S} = μ_{OPM2} | 0.02 | <0.001 |
| UCHL5, μ_{MM.1S} = μ_{OPM2} | 0.042 | <0.001 |
| USP14, μ_{MM.1S} = μ_{OPM2} | 0.022 | <0.001 |
| Rpn11, μ_{MM.1S} = μ_{OPM2} | 0.038 | <0.001 |
| UCHL1, μ_{MM.1S} = μ_{OPM2} | 0.013 | <0.001 |

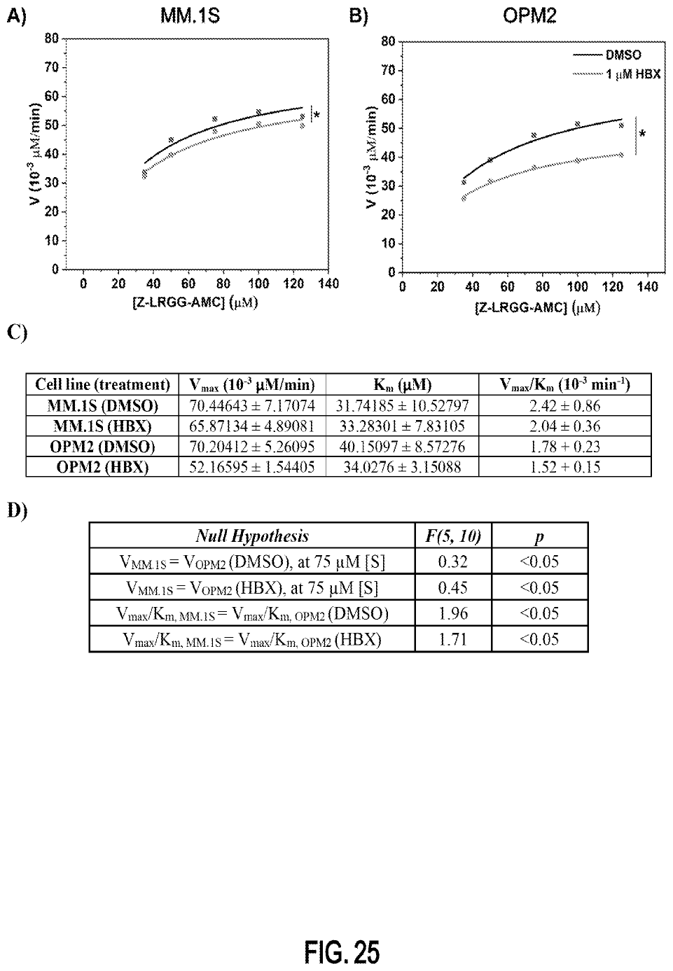

To further validate the differential gene expression of DUBs between MM.1S and OPM2, an enzyme assay was performed in the presence of a commercially available selective DUB inhibitor for USP7 (HBX41108). USP7 was selected based on the observed differences in gene expression from the mRNA screen (FIG. 18). MM.1S and OPM2 cells were treated separately with 1 μM HBX 41108 (a small-molecule inhibitor of USP7/HAUSP ubiquitin protease that stabilizes and activates p53 in cells) or DMSO (control) for 24 h prior and then interrogated for DUB activity using the same fluorometry approach described herein.⁴¹ Results and statistical analysis obtained from this experiment are presented in FIG. 25, from where Vₘₐₓ/Kₘ (10-3 min⁻¹) reveal that the kinetic activity of USP7 in MM.1S (2.42±0.86 for DMSO, 2.04±0.36 for HBX) is higher than that of OPM2 (1.78+0.23 for DMSO, 1.52+0.15 for HBX). Further, p<0.005 show that the rate V (04/min) of USP7 kinetics at 75 μM reporter for MM.1S is statistically higher than that of OPM2. This variability in these standard DUB levels between both the cell lines can be a contributing factor to the preferential interaction of the selective bioactive compounds of PMI-5011 (as discussed in FIGS. 16 and 17).

Conclusions

It is demonstrated herein that a botanical extract called PMI-5011, which exhibits antidiabetic properties through regulation of UPS enzymes, exhibited a prominent effect on DUB activity in model multiple myeloma cells. Enzyme-substrate analysis and mathematical modeling revealed that PMI-5011 enhanced DUB activity in both these cell lines without affecting their viability. A heterotropic co-operativity model is demonstrated here for PMI-5011 treatment, where the compound can have bound directly or indirectly to the DUB activity domains to regulate their catalytic behavior. While a similar heterotropic reaction kinetics can also govern the bioactive compounds DMC-1, DMC-2, and DVG and a knock-out extract (KOE), they demonstrated variable concentration- and cell line-dependent effects on DUB regulation. Calculation of reaction constants demonstrated that the KOE enhanced DUB activity in both OPM2 and MM.1S cell lines, whereas DMC-1 and DMC-2 were found to not be the primary compounds to stimulate DUB activity in the MM.1S cells but did greatly enhance DUB activity in OPM2 cells. Similarly, davidigenin inhibited DUB activity in MM.1S cells but enhanced it in OPM2 cells. This indicated that differential regulation of DUB activity occurred when different bioactive compounds were present when compared to single compound. These differences in the observed trends can be explained by the differently regulated DUBs present in MM.1S and OPM2 cell lines. Results from mRNA profiling demonstrated that this cell dependent effect of PMI-5011 and its compounds on OPM2 and MM.1S was due to differential expression of specific DUBs such as USP7, USP15, USP14, Rpn11 in OPM2 and MM.1S. The results provide information on the plant extract's effect on DUB regulation in addition to showing the allosteric nature of DUBs. Without wishing to be bound by theory, PMI-5011 can have therapeutic uses and can be used alongside proteasome inhibitors to suppress proteasome activity.

### REFERENCES

1. Kuhn, D. J.; Chen, Q.; Voorhees, P. M.; Strader, J. S.; Shenk, K. D.; Sun, C. M.; Demo, S. D.; Bennett, M. K.; van Leeuwen, F. W.; Chanan-Khan, A. A.; Orlowski, R. Z., Potent activity of carfilzomib, a novel, irreversible inhibitor of the ubiquitin-proteasome pathway, against preclinical models of multiple myeloma. Blood 2007, 110 (9), 3281-90.
2. Dietel, M.; Sers, C., Personalized medicine and development of targeted therapies: The upcoming challenge for diagnostic molecular pathology. A review. Virchows Archiv: an international journal of pathology 2006, 448 (6), 744-55.
3. Bedard, P. L.; Hansen, A. R.; Ratain, M. J.; Siu, L. L., Tumour heterogeneity in the clinic. Nature 2013, 501 (7467), 355-64.
4. Croce, C. M., Oncogenes and Cancer. New England Journal of Medicine 2008, 358 (5), 502-511.
5. Richardson, P. G.; Baz, R.; Wang, M.; Jakubowiak, A. J.; Laubach, J. P.; Harvey, R. D.; Talpaz, M.; Berg, D.; Liu, G.; Yu, J.; Gupta, N.; Di Bacco, A.; Hui, A. M.; Lonial, S., Phase 1 study of twice-weekly ixazomib, an oral proteasome inhibitor, in relapsed/refractory multiple myeloma patients. Blood 2014, 124 (7), 1038-46.
6. Tobinai, K., Proteasome inhibitor, bortezomib, for myeloma and lymphoma. International journal of clinical oncology 2007, 12 (5), 318-26.
7. Csermely, P.; Agoston, V.; Pongor, S., The efficiency of multi-target drugs: the network approach might help drug design. Trends in Pharmacological Sciences 2005, 26 (4), 178-182.
8. Tian, Z.; D'Arcy, P.; Wang, X.; Ray, A.; Tai, Y.-T.; Hu, Y.; Carrasco, R. D.; Richardson, P.; Linder, S.; Chauhan, D., A novel small molecule inhibitor of deubiquitylating enzyme USP14 and UCHL5 induces apoptosis in multiple myeloma and overcomes bortezomib resistance. Blood 2014, 123 (5), 706-716.
9. D'Arcy, P.; Linder, S., Proteasome deubiquitinases as novel targets for cancer therapy. The international journal of biochemistry & cell biology 2012, 44 (11), 1729-1738.
10. Teyra, J.; Singer, A. U.; Schmitges, F. W.; Jaynes, P.; Lui, S. K. L.; Polyak, M. J.; Fodil, N.; Krieger, J. R.; Tong, J.; Schwerdtfeger, C., Structural and functional characterization of ubiquitin variant inhibitors of USP15. Structure 2019, 27 (4), 590-605. e5.
11. Wu, X.; Yen, L.; Irwin, L.; Sweeney, C.; Carraway, K. L., Stabilization of the E3 ubiquitin ligase Nrdp1 by the deubiquitinating enzyme USPS. Molecular and cellular biology 2004, 24 (17), 7748-7757.
12. Zhao, L.; Wang, X.; Yu, Y.; Deng, L.; Chen, L.; Peng, X.; Jiao, C.; Gao, G.; Tan, X.; Pan, W., OTUB1 protein suppresses mTOR complex 1 (mTORC1) activity by deubiquitinating the mTORC1 inhibitor DEPTOR. Journal of Biological Chemistry 2018, 293 (13), 4883-4892.
13. Nazio, F.; Strappazzon, F.; Antonioli, M.; Bielli, P.; Cianfanelli, V.; Bordi, M.; Gretzmeier, C.; Dengjel, J.; Piacentini, M.; Fimia, G. M., mTOR inhibits autophagy by controlling ULK1 ubiquitylation, self-association and function through AMBRA1 and TRAF6. Nature cell biology 2013, 15 (4), 406.
14. Dao, J.; Dao, T. C.; Tong, D. D.; Wilson, L.; Jordan, M. A.; Gerwick, W., Compositions of botanical extracts for cancer therapy. Google Patents: 2012.
15. Ribnicky, D.; Poulev, A.; Watford, M.; Cefalu, W.; Raskin, I., Antihyperglycemic activity of Tarralin™, an ethanolic extract of Artemisia dracunculus L. Phytomedicine 2006, 13 (8), 550-557.
16. Ribnicky, D. M.; Kuhn, P.; Poulev, A.; Logendra, S.; Zuberi, A.; Cefalu, W. T.; Raskin, I., Improved absorption and bioactivity of active compounds from an anti-diabetic extract of Artemisia dracunculus L. Int. J Pharm 2009, 370 (1-2), 87-92.
17. Wang, Z. Q.; Ribnicky, D.; Zhang, X. H.; Raskin, I.; Yu, Y.; Cefalu, W. T., Bioactives of Artemisia dracunculus L enhance cellular insulin signaling in primary human skeletal muscle culture. Metabolism 2008, 57, S58-S64.
18. Kirk-Ballard, H.; Kilroy, G.; Day, B. C.; Wang, Z. Q.; Ribnicky, D. M.; Cefalu, W. T.; Floyd, Z. E., An ethanolic extract of Artemisia dracunculus L. regulates gene expression of ubiquitin-proteasome system enzymes in skeletal muscle: Potential role in the treatment of sarcopenic obesity. Nutrition 2014, 30 (7-8), S21-S25.
19. Kirk-Ballard, H.; Wang, Z. Q.; Acharya, P.; Zhang, X. H.; Yu, Y.; Kilroy, G.; Ribnicky, D.; Cefalu, W. T.; Floyd, Z. E., An extract of Artemisia dracunculus L. inhibits ubiquitin-proteasome activity and preserves skeletal muscle mass in a murine model of diabetes. PloS one 2013, 8 (2).
20. Logendra, S.; Ribnicky, D. M.; Yang, H.; Poulev, A.; Ma, J.; Kennelly, E. J.; Raskin, I., Bioassay-guided isolation of aldose reductase inhibitors from Artemisia dracunculus. Phytochemistry 2006, 67 (14), 1539-1546.
21. Schmidt, B.; Ribnicky, D. M.; Poulev, A.; Logendra, S.; Cefalu, W. T.; Raskin, I., A natural history of botanical therapeutics. Metabolism 2008, 57, S3-S9.
22. Zuberi, A. R., Strategies for assessment of botanical action on metabolic syndrome in the mouse and evidence for a genotype-specific effect of Russian tarragon in the regulation of insulin sensitivity. Metabolism 2008, 57, S10-S15.
23. Govorko, D.; Logendra, S.; Wang, Y.; Esposito, D.; Komarnytsky, S.; Ribnicky, D.; Poulev, A.; Wang, Z.; Cefalu, W. T.; Raskin, I., Polyphenolic compounds from Artemisia dracunculus L. inhibit PEPCK gene expression and gluconeogenesis in an H4IIE hepatoma cell line. American Journal of Physiology-Endocrinology and Metabolism 2007, 293 (6), E1503-E1510.
24. Yu, Y.; Simmler, C.; Kuhn, P.; Poulev, A.; Raskin, I.; Ribnicky, D.; Floyd, Z. E.; Pauli, G. F., The DESIGNER Approach Helps Decipher the Hypoglycemic Bioactive Principles of Artemisia dracunculus (Russian Tarragon). Journal of Natural Products 2019.
25. Simon, L.; Salam, A. A. A.; Kumar, S. M.; Shilpa, T.; Srinivasan, K.; Byrappa, K., Synthesis, anticancer, structural, and computational docking studies of 3-benzylchroman-4-one derivatives. Bioorganic & medicinal chemistry letters 2017, 27 (23) 5284-5290.
26. Sahtoe, D. D.; Sixma, T. K., Layers of DUB regulation. Trends in biochemical sciences 2015, 40 (8), 456-467.
27. Faggiano, S.; Menon, R. P.; Kelly, G. P.; Todi, S. V.; Scaglione, K. M.; Konarev, P. V.; Svergun, D. I.; Paulson, H. L.; Pastore, A., Allosteric regulation of deubiquitylase activity through ubiquitination. Frontiers in molecular biosciences 2015, 2, 2.
28. Safa, N.; Pettigrew, J. H.; Gauthier, T. J.; Melvin, A. T., Direct measurement of deubiquitinating enzyme activity in intact cells using a protease-resistant, cell-permeable, peptide-based reporter. Biochemical Engineering Journal 2019, 151, 107320.
29. Tuasha, N.; Seifu, D.; Gadisa, E.; Petros, B.; Oredsson, S., Cytotoxicity of selected Ethiopian medicinal plants used in traditional breast cancer treatment against breast-derived cell lines. Journal of Medicinal Plants Research 2019, 13 (9), 188-198.
30. Bhutia, S. K.; Panda, P. K.; Sinha, N.; Praharaj, P. P.; Bhol, C. S.; Panigrahi, D. P.; Mahapatra, K. K.; Saha, S.; Patra, S.; Mishra, S. R., Plant lectins in cancer therapeutics: Targeting apoptosis and autophagy-dependent cell death. Pharmacological research 2019, 144, 8-18.
31. Khazaei, M.; Pazhouhi, M., Antiproliferative effect of Trifolium pratens L. extract in human breast cancer cells. Nutrition and cancer 2019, 71 (1), 128-140.
32. Majolo, F.; Delwing, L. K. d. 0. B.; Marmitt, D. J.; Bustamante-Filho, I. C.; Goettert, M. I., Medicinal plants and bioactive natural compounds for cancer treatment: Important advances for drug discovery. Phytochemistry Letters 2019, 31, 196-207.
33. Wang, J.; Fernandez, A. E.; Tiano, S.; Huang, J.; Floyd, E.; Poulev, A.; Ribnicky, D.; Pasinetti, G. M., An Extract of Artemisia dracunculus L. Promotes Psychological Resilience in a Mouse Model of Depression. Oxidative Medicine and Cellular Longevity 2018, 2018.
34. Komander, D.; Clague, M. J.; Urbé, S., Breaking the chains: structure and function of the deubiquitinases. Nature reviews Molecular cell biology 2009, 10 (8), 550-563.
35. Mevissen, T. E.; Komander, D., Mechanisms of deubiquitinase specificity and regulation. Annual review of biochemistry 2017, 86, 159-192.
36. Sahtoe, D. D.; van Dijk, W. J.; El Oualid, F.; Ekkebus, R.; Ovaa, H.; Sixma, T. K., Mechanism of UCH-L5 activation and inhibition by DEUBAD domains in RPN13 and INO80G. Molecular cell 2015, 57 (5), 887-900.
37. Farshi, P.; Deshmukh, R. R.; Nwankwo, J. O.; Arkwright, R. T.; Cvek, B.; Liu, J.; Dou, Q. P., Deubiquitinases (DUBs) and DUB inhibitors: a patent review. Expert opinion on therapeutic patents 2015, 25 (10), 1191-1208.
38. Bahuguna, A.; Khan, I.; Bajpai, V. K.; Kang, S. C., MTT assay to evaluate the cytotoxic potential of a drug. Bangladesh Journal of Pharmacology 2017, 12 (2), Online: Apr. 8-2017.
39. Tian, Z.; D'Arcy, P.; Wang, X.; Ray, A.; Tai, Y.-T.; Hu, Y.; Carrasco, R. D.; Richardson, P.; Linder, S.; Chauhan, D., A novel small molecule inhibitor of deubiquitylating enzyme USP14 and UCHL5 induces apoptosis in multiple myeloma and overcomes bortezomib resistance. Blood, The Journal of the American Society of Hematology 2014, 123 (5), 706-716.
40. Nikesitch, N.; Lee, J. M.; Ling, S.; Roberts, T. L., Endoplasmic reticulum stress in the development of multiple myeloma and drug resistance. Clinical & translational immunology 2018, 7 (1), e1007.
41. Colland, F.; Formstecher, E.; Jacq, X.; Reverdy, C.; Planquette, C.; Conrath, S.; Trouplin, V.; Bianchi, J.; Aushev, V. N.; Camonis, J., Small-molecule inhibitor of USP7/HAUSP ubiquitin protease stabilizes and activates p53 in cells. Molecular cancer therapeutics 2009, 8 (8), 2286-2295.

### Example 4

### Experiment 1

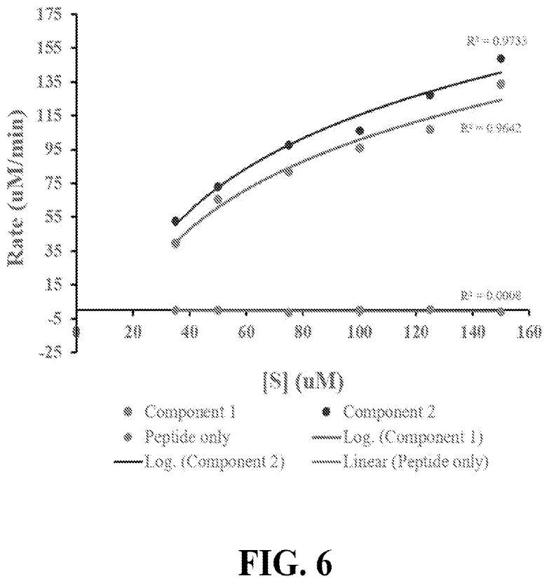

Peptide used was the commercial DUB reporter [Z-LRGG-AMC (SEQ ID NO: 1)] at multiple concentration [35-150 uM]. 1500 μL protein at 10 mg/mL for each set was given. Final protein concentration in micro-wells was 4 mg/mL. Peptide was incubated with component 1 and component 2. Fluorometric readings were taken at different time points in plate reader. Measured at [Em 460/Ex 355]. Fluorescent signals were converted to μM/min using a calibration curve for the AMC fluorophore. Reaction rates (μM/min) were plotted against substrate concentrations [S] (μM) (FIG. 6). Rates were plotted vs. substrate concentration (Z-LRGG-AMC peptide (SEQ ID NO: 1)) The data points look neat without any fluctuations—the triplicate values for each datapoint were consistent. The results of experiment 1 generated clear data points.

### Example 5

### Experiment 2

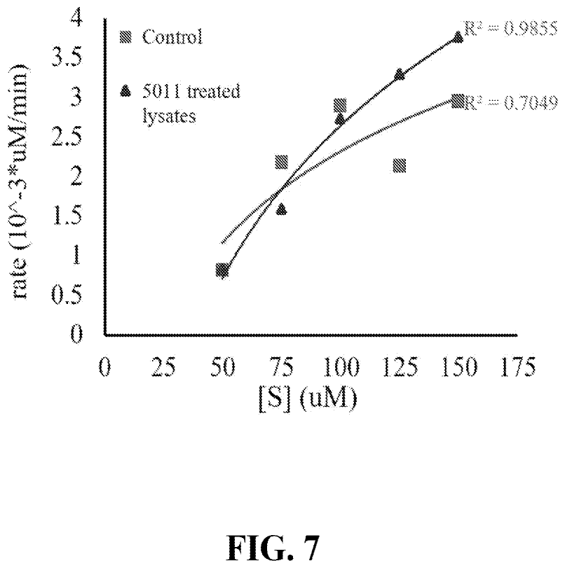

Peptide used was the commercial DUB reporter [Z-LRGG-AMC (SEQ ID NO: 1)] at multiple concentration [50-150 um]. 640 μL protein at 10 mg/mL for each set was given. Final protein concentration in micro-wells was 4 mg/mL. Peptide was incubated with control, 5011 treated, 25 uM IU1, 50 uM IU1 and KOE in DUB assay buffer. Fluorometric readings were taken at different time points in plate reader. Measured at [Em 460/Ex 355]. Fluorescent signals were converted to μM/min using a calibration curve for the AMC fluorophore. Rates were plotted vs. substrate concentration (Z-LRGG-AMC peptide (SEQ ID NO: 1)) (FIG. 7).

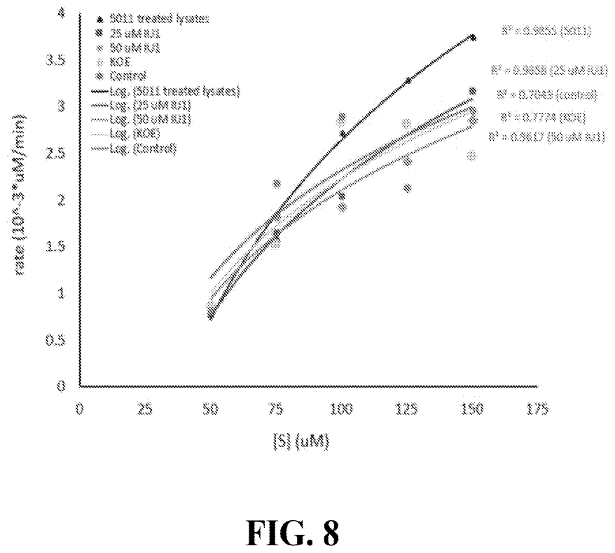

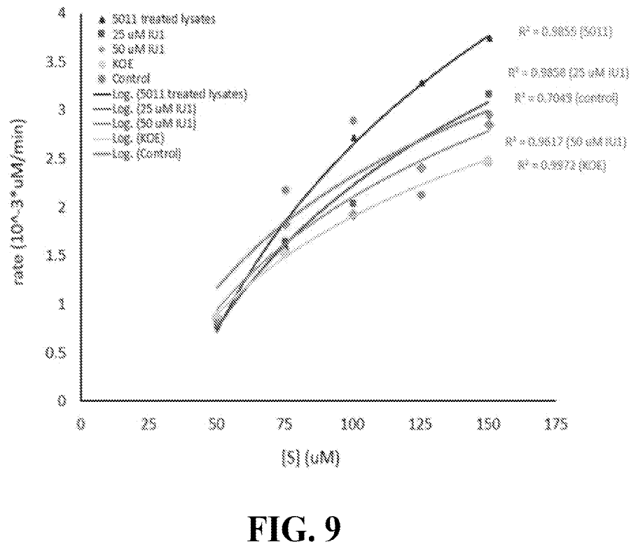

The two concentrations of USP14 inhibitor-25 μM and 50 μM IU1 seems to inhibit the DUB activity (decreased signal) (FIG. 8). Comparing to control, 25 μM IU1 appears a less of a concentration to inhibit DUB activity ˜550 μM IUI does better inhibition of DUB activity. For better curve fitting (FIG. 9), values at 100 μM and 125 μM [S] for KOE and 125 μM [S] for 25 uM IU1 were ignored. KOE, the inactive part of 5011 inhibits DUB activity.

Conclusion

The experiment will be repeated to check control values and to make sure to what we are seeing with KOE and DUB inhibitors is right. Additionally, 35 μM [S] and 175 μM [S] data points can be collected for better understanding.

### Example 6

### Experiment 3

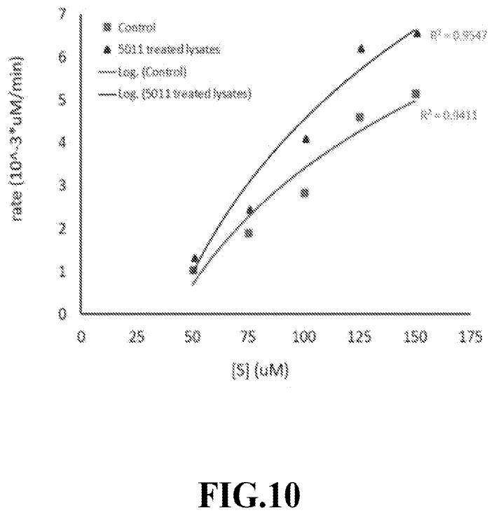

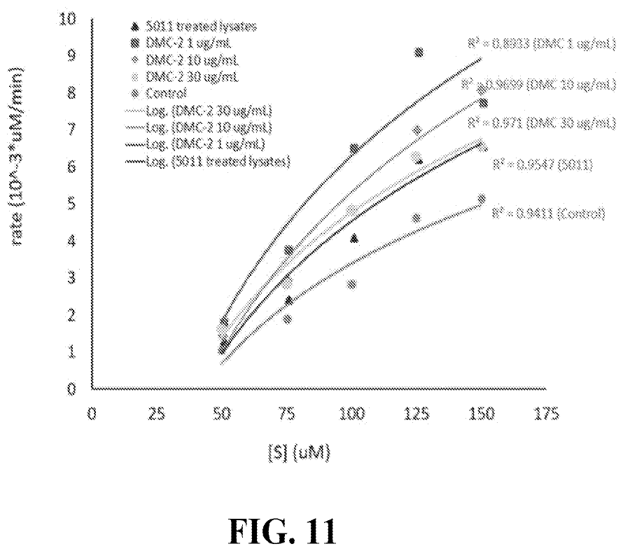

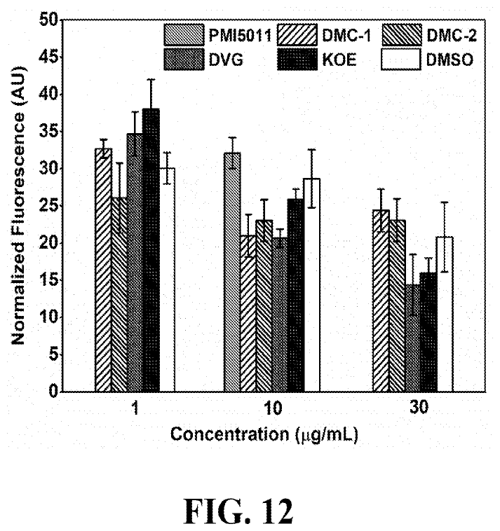

Peptide used was the commercial DUB reporter [Z-LRGG-AMC (SEQ ID NO: 1)] at multiple concentrations [50-150 uM]. 1100 uL protein at 6 mg/mL for each set was given. Final protein concentration in micro-wells was 4 mg/mL. Peptide was incubated with control, 5011 treated, 1 ug/mL DMC-2, 10 ug/mL DMC-2, and 30 ug/mL DMC-2 in DUB assay buffer. Fluorometric readings were taken at different time points in plate reader. Measured at [Em 460/Ex 355]. Fluorescent signals were converted to μM/min using a calibration curve for the AMC fluorophore. Rates were plotted vs. substrate concentration (Z-LRGG-AMC peptide (SEQ ID NO: 1)) (FIG. 10). Without wishing to be bound by theory, the experiment indicates that 5011 enhances DUB activity (compare with control). The curves are not perfect logarithmic curves but we can observe the trend. The results with different concentrations of DMC-2 were further plotted. When compared with control: the 5011 treatment enhances DUB activity, DMC-2 being the active part of 5011, also enhances the DUB activity. From FIG. 11, the lowest concentration of DMC2-1 μg/mL has most effect on DUB activity when compared to 30 μg/mL. Without wishing to be bound by theory, some data fluctuation with DMC-2 can occur in the 1 μg/mL range.

### Example 7

Extraction and Synthesis of PMI-5011, KOE and Selected Bioactive Compounds

The ethanolic extract of Artemisia dracunculus L, PMI-5011 and its knockout extract, KOE were obtained from plants grown hydroponically in greenhouses under uniform and strictly controlled conditions, thereby standardizing the plants for their phytochemical content. Detailed information about quality control, preparation and extraction of PMI-5011 and KOE has been previously reported.¹ The selected bioactive compounds of PMI-5011 (DMC-1, DMC-2, and davidigenin) were chemically synthesized as described herein using modified procedures previously reported.²

Synthesis of 4-(ethoxymethoxy)benzaldehyde (1)

To a stirred solution of 4-hydroxybenzaldehyde (1 eq, 5 g, 40.943 mmol) in acetone (30 mL) was added K₂CO₃ (2 eq, 11.32 g, 81.886 mmol) followed by chloromethyl ethyl ether (1.2 eq, 5.7 mL, 49.132 mmol). The reaction mixture was stirred at room temperature overnight. The reaction was quenched with water (150 mL) and extracted with EtOAc (3×100 mL). The organic layers were combined and washed with water (2×100 mL) then dried over anhydrous Na₂SO₄ and filtered. The solvent was removed in vacuo to yield the crude product. The title compound was purified by flash chromatography using 230-400 mesh silica gel (120 g) and eluted with hexane:ethyl acetate (7:1) to afford compound 1 as a clear oil (4.192 g, 57%).

### Synthesis of 1-(4-(ethoxymethyl)-2-hydroxyphenyl)ethan-1-one (2)

To a stirred solution of 2,4-dihydroxyacetophenone (1 eq, 5 g, 32.862 mmol) in acetone (30 mL) was added K₂CO₃ (2 eq, 9.084 g, 65.725 mmol) followed by chloromethyl ethyl ether (1.2 eq, 4.660 g, 39.434 mmol). The reaction mixture was stirred at room temperature overnight. The reaction was quenched with water (150 mL) and extracted with EtOAc (3×100 mL). The organic layers were combined and washed with water (2×100 mL) then dried over anhydrous Na₂SO₄ and filtered. The solvent was removed in vacuo to yield the crude product. The title compound was purified by flash chromatography using 230-400 mesh silica gel (150 g) and eluted with hexane:ethyl acetate (7:1) to afford compound 2 as a clear oil (1.966 g, 31%).

### Synthesis of (E)-3-(4-(ethoxymethoxy)phenyl)-1-(2-hydroxy-4-methoxyphenyl)prop-2-en-1-one (3)

Compound 1 (1 eq, 1 g, 5.55 mmol), 2-hydroxy-4-methoxyacetophenone (2 eq, 1.84 g, 11.1 mmol) and 5M KOH in ethanol (8.3 eq, 9.21 mL) were placed in a 10-20 mL microwave reaction vial and sealed. The vial was placed in a Biotage Initiator microwave reactor (Biotage, LLC; Charlotte, N.C.) and the reaction was carried out at 85° C. for 15 minutes with a 30 second pre-stir. After completion, the reaction mixture was transferred to a separatory funnel and extracted with ethyl acetate (3×50 mL). The ethyl acetate layer was washed with water (2×50 mL) and brine (1×50 mL). The organic layer was dried over anhydrous Na₂SO₄, filtered and concentrated in vacuo. The residue was dissolved in methanol (50 mL) and placed in the freezer overnight. The yellow solid was filtered and dried to yield compound 3 (1.032 g, 57%).

### Synthesis of (E)-1-(2-hydroxy-4-methoxyphenyl)-3-(4-hydroxyphenyl)prop-2-en-1-one (4)

Compound 3 (300 mg, 0.914 mmol) was dissolved in ethanol (12 mL) and placed in a 10-20 mL microwave reaction vial along with 6N HCL (5 mL). The vial was sealed and placed in a Biotage Initiator microwave reactor (Biotage, LLC; Charlotte, N.C.). The reaction was carried out at 70° C. for 1.5 minutes with a 30 second pre-stir. After completion, the reaction mixture was neutralized with NaHCO₃ to pH 7 and transferred to a separatory funnel and extracted with ethyl acetate (2×80 mL). The ethyl acetate layer was washed with water (2×50 mL) and brine (2×60 mL). The organic layer was dried over anhydrous Na₂SO₄ and concentrated in vacuo to yield compound 4 as a yellow solid (415 mg, 84%).

### Synthesis of 1-(2-hydroxy-4-methoxyphenyl)-3-(4-hydroxyphenyl)propan-1-one (5)

To a stirred solution of compound 4 (1 eq, 409 mg, 1.51 mmol) in ethanol (50 ml), 10% Pd/C (0.1 eq, 161 mg, 0.151 mmol) was added. Hydrogen gas was generated by slowly dripping 6M H₂SO₄ (30 mL) over 15g crushed Zn. The hydrogen gas was bubbled into the reaction mixture for 1 hour. After completion, the solution was filtered through celite and the clear liquid was concentrated in vacuo to yield a white solid (383 mg). The title compound was purified by flash chromatography using 230-400 mesh silica gel (20 g) and eluted with DCM to afford DMC-1 as a white solid (245 mg, 64%). HRMS (ESI) calc'd for C₁₆H₁₅O₄ [M−H]⁻ 271.0970. found 271.0973.

### Synthesis of (E)-1-(4-(ethoxymethoxy)-2-hydroxyphenyl)-3-(4-methoxyphenyl)prop-2-en-1-one (6)

Compound 2 (1 eq, 500 mg, 2.378 mmol), p-anisaldehyde (2 eq, 648 mg, 4.757 mmol) and 5M KOH in ethanol (8.3 eq, 3.95 mL) were placed in a 10-20 mL microwave reaction vial and sealed. The vial was placed in a Biotage Initiator microwave reactor (Biotage, LLC; Charlotte, N.C.) and the reaction was carried out at 85° C. for 15 minutes with a 30 second pre-stir. After completion, the reaction mixture was transferred to a separatory funnel and extracted with ethyl acetate (3×50 mL). The ethyl acetate layer was washed with water (2×50 mL) and brine (1×50 mL). The organic layer was dried over anhydrous Na₂SO₄, filtered and concentrated in vacuo. The residue was dissolved in methanol (50 mL) and placed in the freezer overnight. The yellow solid was filtered and dried to yield compound 6 (381 mg, 49%).

### Synthesis of (E)-1-(2,4-dihydroxyphenyl)-3-(4-methoxyphenyl)prop-2-en-1-one (7)

Compound 6 (300 mg, 0.914 mmol) was dissolved in ethanol (12 mL) and placed in a 10-20 mL microwave reaction vial along with 6N HCL (5 mL). The vial was sealed and placed in a Biotage Initiator microwave reactor (Biotage, LLC; Charlotte, N.C.). The reaction was carried out at 70° C. for 1.5 minutes with a 30 second pre-stir. After completion, the reaction mixture was neutralized with NaHCO₃ to pH 7 and transferred to a separatory funnel and extracted with ethyl acetate (2×80 mL). The ethyl acetate layer was washed with water (2×50 mL) and brine (2×60 mL). The organic layer was dried over anhydrous Na₂SO₄ and concentrated in vacuo to yield compound 7 (233 mg, 95%) as a yellow solid.

### Synthesis of 1-(2,4-dihydroxyphenyl)-3-(4-methoxyphenyl)propan-1-one (8)

To a stirred solution of compound 4 (1 eq, 210 mg, 0.777 mmol) in ethanol (50 ml), 10% Pd/C (0.1 eq, 83 mg, 0.0777 mmol) was added. Hydrogen gas was generated by slowly dripping 6M H₂SO₄ (30 mL) over crushed Zn (15 g). The hydrogen gas was bubbled into the reaction mixture for 1 hour. After completion, the solution was filtered through celite and the clear liquid was concentrated in vacuo to yield a white solid (203 mg). The title compound was purified by flash chromatography using 230-400 mesh silica gel (20 g) and eluted with DCM to afford DMC-2 as a white solid (133 mg, 66%). HRMS (ESI) calc'd for C₁₆H₁₅O₄ [M−H]⁻ 271.0970. found 271.0979.

### Synthesis of (E)-1-(4-(ethoxymethoxy)-2-hydroxyphenyl)-3-(4-(ethoxymethoxy)phenyl)prop-2-en-1-one (9)

Compound 2 (1 eq, 500 mg, 2.378 mmol), compound 1 (2 eq, 866 mg, 4.806 mmol) and 5M KOH in ethanol (8.3 eq, 3.95 mL) were placed in a 10-20 mL microwave reaction vial and sealed. The vial was placed in a Biotage Initiator microwave reactor (Biotage, LLC; Charlotte, N.C.) and the reaction was carried out at 85° C. for 15 minutes with a 30 second pre-stir. After completion, the reaction mixture was transferred to a separatory funnel and extracted with ethyl acetate (3×50 mL). The ethyl acetate layer was washed with water (2×50 mL) and brine (1×50 mL). The organic layer was dried over anhydrous Na₂SO₄, filtered and concentrated in vacuo. The residue was dissolved in methanol (50 mL) and placed in the freezer overnight. The yellow solid was filtered and dried to yield compound 9 (417 mg, 47%).

### Synthesis of 1-(4-(ethoxymethoxy)-2-hydroxyphenyl)-3-(4-(ethoxymethoxy)phenyl)propan-1-one (10)

To a stirred solution of compound 9 (1 eq, 321 mg, 0.862 mmol) in ethanol (100 ml), 10% Pd/C (0.1 eq, 92 mg, 0.0865 mmol) was added. Hydrogen gas was generated by slowly dripping 6M H₂SO₄ (30 mL) over crushed Zn (15 g). The hydrogen gas was bubbled into the reaction mixture for 1 hour. After completion, the solution was filtered through celite and the clear liquid was concentrated in vacuo to yield compound 10 as a white solid (306 mg, 96%).

### Synthesis of 1-(2,4-dihydroxyphenyl)-3-(4-hydroxyphenyl)propan-1-one (11)

Compound 10 (306 mg, 0.817 mmol) was dissolved in ethanol (12 mL) and placed in a 10-20 mL microwave reaction vial along with 6N HCl (5 mL). The vial was sealed and placed in a Biotage Initiator microwave reactor (Biotage, LLC; Charlotte, N.C.). The reaction was carried out at 70° C. for 1.5 minutes with a 30 second pre-stir. The reaction mixture was neutralized with NaHCO₃ to pH 7 and transferred to a separatory funnel and extracted with ethyl acetate (2×80 mL). The ethyl acetate layer was washed with water (2×50 mL) and brine (2×60 mL). The organic layer was dried over anhydrous Na₂SO₄, filtered and concentrated in vacuo to yield a white solid (224 mg). The title compound was purified by flash chromatography using 230-400 mesh silica gel (18 g) and eluted with DCM: ethyl acetate (19:1) to afford davidigenin as white solid (94 mg, 45%). HRMS (ESI) calc'd for C1411304 [M−H]⁻ 257.0814. found 257.0827.

### REFERENCES CITED IN THIS EXAMPLE

1. (a) Schmidt, B.; Ribnicky, D. M.; Poulev, A.; Logendra, S.; Cefalu, W. T.; Raskin, I., A natural history of botanical therapeutics. Metabolism 2008, 57, S3-S9; (b) Ribnicky, D.; Poulev, A.; Watford, M.; Cefalu, W.; Raskin, I., Antihyperglycemic activity of Tarralin™, an ethanolic extract of Artemisia dracunculus L. Phytomedicine 2006, 13 (8), 550-557; (c) Ribnicky, D. M.; Kuhn, P.; Poulev, A.; Logendra, S.; Zuberi, A.; Cefalu, W. T.; Raskin, I., Improved absorption and bioactivity of active compounds from an anti-diabetic extract of Artemisia dracunculus L. Int. J. Pharm 2009, 370 (1-2), 87-92; (d) Zuberi, A. R., Strategies for assessment of botanical action on metabolic syndrome in the mouse and evidence for a genotype-specific effect of Russian tarragon in the regulation of insulin sensitivity. Metabolism 2008, 57, S10-S15; (e) Logendra, S.; Ribnicky, D. M.; Yang, H.; Poulev, A.; Ma, J.; Kennelly, E. J.; Raskin, I., Bioassay-guided isolation of aldose reductase inhibitors from Artemisia dracunculus. Phytochemistry 2006, 67 (14), 1539-1546; (f) Govorko, D.; Logendra, S.; Wang, Y.; Esposito, D.; Komarnytsky, S.; Ribnicky, D.; Poulev, A.; Wang, Z.; Cefalu, W. T.; Raskin, I., Polyphenolic compounds from Artemisia dracunculus L. inhibit PEPCK gene expression and gluconeogenesis in an H4IIE hepatoma cell line. American Journal of Physiology-Endocrinology and Metabolism 2007, 293 (6), E1 503-E1510; (g) Yu, Y.; Simmler, C.; Kuhn, P.; Poulev, A.; Raskin, I.; Ribnicky, D.; Floyd, Z. E.; Pauli, G. F., The DESIGNER Approach Helps Decipher the Hypoglycemic Bioactive Principles of Artemisia dracunculus (Russian Tarragon). Journal of Natural Products 2019.
2. Simon, L.; Salam, A. A. A.; Kumar, S. M.; Shilpa, T.; Srinivasan, K.; Byrappa, K., Synthesis, anticancer, structural, and computational docking studies of 3-benzylchroman-4-one derivatives. Bioorganic & medicinal chemistry letters 2017, 27 (23) 5284-5290.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of treating a disease or disorder characterized by aberrations in the ubiquitin proteasome system (UPS), the method comprising administering to a subject a therapeutically effective amount of a botanical extract or components thereof, wherein the botanical extract is isolated from Artemisia spp.

2. The method of claim 1, wherein the disease or disorder comprises a cell proliferative disease or disorder, a muscle wasting disease or disorder, a renal disease or disorder, an inflammatory disease or disorder, a neurodegenerative disease or disorder, or a ubiquitin-proteasome system (UPS)-associated disease or disorder.

3. The method of claim 2, wherein the cell proliferative disease or disorder comprises cancer.

4. The method of claim 3, wherein the cancer comprises multiple myeloma.

5. The method of claim 2, wherein the muscle wasting disease or disorder comprises sarcopenia.

6. The method of claim 2, wherein the UPS-associated disease or disorder is characterized by aberrations in the ubiquitin-proteasome system (UPS).

7. The method of claim 1, wherein the botanical extract is isolated from Artemisia dracunculus L.

8. The method of claim 1, wherein the botanical extract comprises PMI5011 or a component thereof.

9. The method of claim 7, wherein the component comprises DMC-1, DMC-2, davidigenin, sakuranetin, or 6-demethoxycapillarisin.

10. The method of claim 1, wherein the botanical extract comprises a knockout extract (KOE).

11. The method of claim 9, wherein the KOE has reduced levels of at least one component relative to the normal extract.

12. The method of claim 10, wherein the KOE has reduced levels of at least one of DMC-1 or DMC-2, relative to the normal extract.

13. The method of claim 12, wherein the KOE does not comprise DMC-1 or DMC-2.

14. The method of claim 12, wherein the KOE does not comprise DMC-1 and DMC-2.

15. The method of claim 1, wherein the botanical extract comprises an alcoholic extract.

16. The method of claim 15, wherein the alcoholic extract comprises an ethanolic extract.

17. The method of claim 1, wherein the botanical extract modulates the activity of at least one deubiquitinating enzyme (DUB).

18. The method of claim 1, wherein the botanical extract is administered to a subject orally, intravenously, sub-cutaneously, or transdermally.

19. The method of claim 1, further comprising administering to a subject in need thereof one or more additional active agents.

20. The method of claim 19, wherein the one or more additional active agents comprises one or more anti-cancer agents, one or more anti-inflammatory agents, or one or more neuroprotective agents.

21. The method of claim 20, wherein the one or more anti-cancer agents comprises Marizomib, Ixazomib, Borezomib, or Carfilzomib.

22. A therapeutic preparation comprising a botanical extract isolated from Artemisia spp., wherein the botanical extract modulates the activity of the UPS.

23. The therapeutic preparation of claim 22, wherein the botanical extract modulates the activity of at least one deubiquitinating enzyme (DUB).

24. The therapeutic preparation of claim 22, wherein the botanical extract is isolated from Artemisia dracunculus L.

25. The therapeutic preparation of claim 22, wherein the botanical extract comprises DMC-1, DMC-2, davidigenin, sakuranetin, or 6-demethoxycapillarisin.

26. The therapeutic preparation of claim 22, wherein the botanical extract comprises a knockout extract (KOE).

27. The therapeutic preparation of claim 26, wherein the KOE has reduced levels of at least one component relative to the normal extract.

28. The therapeutic preparation of claim 27, wherein the KOE has reduced levels of at least one of DMC-1 or DMC-2 relative to the normal extract.

29. The therapeutic preparation of claim 27, wherein the KOE does not comprise at least one of DMC-1 or DMC-2.

30. The therapeutic preparation of claim 28, wherein the KOE does not comprise DMC-1 and DMC-2.

31. The therapeutic preparation of claim 22, wherein the botanical extract comprises an alcoholic extract.

32. The therapeutic preparation of claim 31, wherein the alcoholic extract comprises an ethanolic extract.

33. A drug-screening method, the method comprising:
culturing a population of cells;
incubating the population of cells with a botanical extract or component thereof, wherein the botanical extract is isolated from Artemisia spp; and
determining the effect of the botanical extract or component thereof on UPS activity.

34. The method of claim 33, further comprising a step of obtaining a population of cells from a subject.

35. The method of claim 33, further comprising a step of culturing a population of control cells.

36. The method of claim 33, further comprising a step of administering the botanical extract or component thereof to a subject.

37. The method of claim 33, wherein the population of cells comprises a population of cancer cells.

38. The method of claim 37, wherein the cancer cells comprises MM.1S cells or OPM2 cells.

39. The method of claim 33, wherein the population of cells are incubated with an amount of the botanical extract for a period of time.

40. The method of claim 33, wherein the UPS activity comprises DUB activity.


SEQUENCE LISTING (15 sequences)

SEQ ID NO: 1 - protein, 4 aa, Artificial Sequence
  Description of Artificial Sequence Synthetic peptide
LRGG

SEQ ID NO: 2 - DNA, 21 bp, Artificial Sequence
  Description of Artificial Sequence Synthetic primer
tcaaagatggtgatgcccagt

SEQ ID NO: 3 - DNA, 20 bp, Artificial Sequence
  Description of Artificial Sequence Synthetic primer
ctgttcaaccacctttcgtg

SEQ ID NO: 4 - DNA, 20 bp, Artificial Sequence
  Description of Artificial Sequence Synthetic primer
tacaccagggcgagcatttt

SEQ ID NO: 5 - DNA, 22 bp, Artificial Sequence
  Description of Artificial Sequence Synthetic primer
ggatgagacatattaccgggct

SEQ ID NO: 6 - DNA, 21 bp, Artificial Sequence
  Description of Artificial Sequence Synthetic primer
agtttcgccagacagcaaatg

SEQ ID NO: 7 - DNA, 21 bp, Artificial Sequence
  Description of Artificial Sequence Synthetic primer
gggttcctctgcaagttgtct

SEQ ID NO: 8 - DNA, 20 bp, Artificial Sequence
  Description of Artificial Sequence Synthetic primer
accctcagccaaaactgtct

SEQ ID NO: 9 - DNA, 20 bp, Artificial Sequence
  Description of Artificial Sequence Synthetic primer
tctcaaggcacctgcatacc

SEQ ID NO: 10 - DNA, 22 bp, Artificial Sequence
  Description of Artificial Sequence Synthetic primer
aaacaagccatctatccaggca

SEQ ID NO: 11 - DNA, 20 bp, Artificial Sequence
  Description of Artificial Sequence Synthetic primer
gggtcctgcttgccaacatt

SEQ ID NO: 12 - DNA, 20 bp, Artificial Sequence
  Description of Artificial Sequence Synthetic primer
aaccccgagatgctgaacaa

SEQ ID NO: 13 - DNA, 20 bp, Artificial Sequence
  Description of Artificial Sequence Synthetic primer
tctacccgacattggccttc

SEQ ID NO: 14 - DNA, 20 bp, Artificial Sequence
  Description of Artificial Sequence Synthetic primer
tggcgtcgtgattagtgatg

SEQ ID NO: 15 - DNA, 22 bp, Artificial Sequence
  Description of Artificial Sequence Synthetic primer
gacgttcagtcctgtccataat